SEQUENCE LISTING (14 sequences)

SEQ ID NO: 1 - protein, 174 aa, Bos taurus
TPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPTQGAMPTFTSAFQRRAGGVLVASQLHRFLELAYRGLRYLAEP

SEQ ID NO: 2 - protein, 175 aa, Bos taurus
MTPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPTQGAMPTFTSAFQRRAGGVLVASQLHRFLELAYRGLRYLAEP

SEQ ID NO: 3 - protein, 175 aa, Bos taurus
  SITE 9: note = misc_feature - wherein Xaa is para-acetylphenylalanine (pAF)
MTPLGPARXLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPTQGAMPTFTSAFQRRAGGVLVASQLHRFLELAYRGLRYLAEP

SEQ ID NO: 4 - protein, 175 aa, Bos taurus
  SITE 63: note = misc_feature - wherein Xaa is para-acetylphenylalanine (pAF)
MTPLGPARSLPQSFLLKCLEQVRKIQADGAELQERLCAAHKLCHPEELMLLRHSLGIPQAPLXSCSSQSLQLTSCLNQLHGGLFLYQGLLQALAGISPELAPTLDTLQLDVTDFATNIWLQMEDLGAAPAVQPTQGAMPTFTSAFQRRAGGVLVASQLHRFLELAYRGLRYLAEP

SEQ ID NO: 5 - protein, 175 aa, Bos taurus
  SITE 70: note = misc_feature - wherein Xaa is para-acetylphenylalanine (pAF)
MTPLGPARSLPQSFLLKCLEQVRKIQADGAELQERLCAAHKLCHPEELMLLRHSLGIPQAPLSSCSSQSXQLTSCLNQLHGGLFLYQGLLQALAGISPELAPTLDTLQLDVTDFATNIWLQMEDLGAAPAVQPTQGAMPTFTSAFQRRAGGVLVASQLHRFLELAYRGLRYLAEP

SEQ ID NO: 6 - protein, 175 aa, Bos taurus
  SITE 126: note = misc_feature - wherein Xaa is para-acetylphenylalanine (pAF)
MTPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLXAAPAVQPTQGAMPTFTSAFQRRAGGVLVASQLHRFLELAYRGLRYLAEP

SEQ ID NO: 7 - protein, 175 aa, Bos taurus
  SITE 134: note = misc_feature - wherein Xaa is para-acetylphenylalanine (pAF)
MTPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPXQGAMPTFTSAFQRRAGGVLVASQLHRFLELAYRGLRYLAEP

SEQ ID NO: 8 - protein, 175 aa, Bos taurus
  SITE 137: note = misc_feature - wherein Xaa is para-acetylphenylalanine (pAF)
MTPLGPARSLPQSFLLKCLEQVRKIQADGAELQERLCAAHKLCHPEELMLLRHSLGIPQAPLSSCSSQSLQLTSCLNQLHGGLFLYQGLLQALAGISPELAPTLDTLQLDVTDFATNIWLQMEDLGAAPAVQPTQGXMPTFTSAFQRRAGGVLVASQLHRFLELAYRGLRYLAEP

SEQ ID NO: 9 - protein, 175 aa, Bos taurus; identical to SEQ ID NO: 3
  SITE 9: note = misc_feature - wherein Xaa is para-acetylphenylalanine (pAF) linked to a poly(ethylene glycol) moiety

SEQ ID NO: 10 - protein, 175 aa, Bos taurus; identical to SEQ ID NO: 4
  SITE 63: note = misc_feature - wherein Xaa is para-acetylphenylalanine (pAF) linked to a poly(ethylene glycol) moiety

SEQ ID NO: 11 - protein, 175 aa, Bos taurus; identical to SEQ ID NO: 5
  SITE 70: note = misc_feature - wherein Xaa is para-acetylphenylalanine (pAF) linked to a poly(ethylene glycol) moiety

SEQ ID NO: 12 - protein, 175 aa, Bos taurus; identical to SEQ ID NO: 6
  SITE 126: note = misc_feature - wherein Xaa is para-acetylphenylalanine (pAF) linked to a poly(ethylene glycol) moiety

SEQ ID NO: 13 - protein, 175 aa, Bos taurus; identical to SEQ ID NO: 7
  SITE 134: note = misc_feature - wherein Xaa is para-acetylphenylalanine (pAF) linked to a poly(ethylene glycol) moiety

SEQ ID NO: 14 - protein, 175 aa, Bos taurus; identical to SEQ ID NO: 8
  SITE 137: note = misc_feature - wherein Xaa is para-acetylphenylalanine (pAF) linked to a poly(ethylene glycol) moiety

(19) United States
(12) Patent Application Publication
(10) Pub. No.: US 2025/0134960 A1
(43) Pub. Date: May 1, 2025

(54) FORMULATIONS FOR BOVINE GRANULOCYTE COLONY STIMULATING FACTOR AND VARIANTS THEREOF

(71) Applicant: ELANCO US INC., Greenfield, IN (US)

(72) Inventors: Alan Voskamp KLOTZ, Indianapolis, IN (US); Catherine Ngan KHA, Raleigh, NC (US); Juan DAVAGNINO, Durham, NC (US)

(21) Appl. No.: 18/916,062

(22) Filed: Oct. 15, 2024

Related U.S. Application Data
(63) Continuation of application No. 17/591,553, filed on Feb. 2, 2022, now Pat. No. 12,138,296.
Application No. 17/591,553 is a continuation of application No. 13/239,493, filed on Sep. 22, 2011, now Pat. No. 11,273,202.
(60) Provisional application No. 61/385,629, filed on Sep. 23, 2010.

(51) Int. Cl. A61K 38/19; C07K 14/535

(52) U.S. Cl. CPC A61K 38/193; C07K 14/535

(57) ABSTRACT

This invention provides stable aqueous formulations comprising a bG-CSF polypeptide or a variant thereof, a buffer substance, and an excipient, wherein said formulation is substantially free of polyoxyethylene (20) sorbitan monolaurate. The invention also provides methods of using, a lyophilized or powdered form of, and processes for, preparing the formulation.

### CROSS-REFERENCE TO RELATED APPLICATIONS

This application is a continuation of U.S. application Ser. No. 17/591,553, filed on Feb. 2, 2022, which is a continuation of U.S. application Ser. No. 13/239,493, filed on Sep. 22, 2011, and claims the benefit of U.S. Provisional Application No. 61/385,629 filed on Sep. 23, 2010, the disclosures of all of which are incorporated by reference herein in their entirety.

Granulocyte Colony Stimulating Factor (G-CSF) is a member of the growth hormone supergene family. G-CSF stimulates the proliferation of specific bone marrow precursor cells and their differentiation into granulocytes. Furthermore, G-CSF is a potent stimulus for neutrophil proliferation and maturation in vivo (Cohen et al., Proc. Natl. Acad. Sci. 1987; 84:2484-2488; see also Heidari et al., Vet. Immol. Imunopathol. 2001; 81:45-57). G-CSF is also capable of inducing functional activation or “priming” of mature neutrophils in vitro (Weisbart, R. H. et al., Annals of Internal Medicine 1989; 110:297-303). G-CSF has been shown to prime human granulocytes and enhance superoxide release stimulated by the chemotactic peptide N-formyl-methionyl-leucyl-phenalalanine (S. Kitagawa, et al., Biochem. Biophys. Res. Commun. 1987; 144:1143-1146, and C. F. Nathan, Blood 1989; 74:301-306), and to activate human neutrophil IgA mediated phagocytosis (Weisbart, R. H., et al., Nature 1988; 332: 647-649).

G-CSF has been found to be useful in the treatment of indications where an increase in neutrophils will provide benefits. G-CSF is also useful alone, or in combination with other compounds (such as other cytokines) for growth or expansion of cells in culture, for example, for bone marrow transplants.

The cDNA cloning and expression of recombinant human G-CSF (hG-CSF) has been described, and the recombinant hG-CSF exhibits most, if not all, of the biological properties of the native molecule (Souza, L. et al., Science 232, 61-65 (1986)). Sequence analysis of the cDNA and genomic DNA clones has allowed the deduction of the amino acid sequence and reveals that the protein is 204 amino acids long with a signal sequence of 30 amino acids. The mature protein is 174 amino acids long and possesses no potential N-linked glycosylation sites but several possible sites for O-linked glycosylation.

Pharmaceutical preparations containing hG-CSF are known in the art and include numerous formulations. For example, various formulations of hG-CSF are described in Piedmonte et al., Advanced Drug Delivery Reviews, 60: 50-58 (2008), Herman et al., in Formulation, Characterization, and Stability of Protein Drugs, Rodney Pearlman and Y. John Wang, eds., Plenum Press, New York (1996), U.S. Pat. No. 5,919,757 to Michaelis et al., and U.S. Pat. No. 6,908,610 to Sato et al. Traditionally, surfactants are included in hG-CSF formulations and may protect hG-CSF at potentially destabilizing interfaces, against surfaces encountered during processing, and against the alteration of its conformational stability.

The cDNA cloning and expression of recombinant bovine G-CSF (bG-CSF) has also been described. For example, the polynucleotide and polypeptide sequence of mature bG-CSF is presented in U.S. Pat. No. 5,849,883, which also describes methods to clone, isolate, and purify the polypeptide and analogs thereof. Mature bG-CSF is 174 amino acids in length and has 82% homology to hG-CSF. Heidari et al., supra, describe the expression, purification, and biological activities of bG-CSF.

Administration of bG-CSF to cattle can provide therapeutic benefits. Accordingly, a pharmaceutical formulation containing bG-CSF is desirable to utilize its therapeutic potential. However, bG-CSF pharmaceutical formulations developed according to traditional methods known in the art result in undesirable product properties, such as aggregation and destabilization of the bG-CSF polypeptide and/or the formulation.

Therefore, there exists a need for a stable bG-CSF pharmaceutical formulation with desirable properties, such as minimal product aggregation and destabilization properties. Accordingly, the present invention provides stable aqueous pharmaceutical formulations with a bG-CSF polypeptide or a variant thereof which exhibit desirable properties and provide related advantages as well.

This invention provides stable aqueous formulations comprising a bG-CSF polypeptide or a variant thereof, a buffer substance, and an excipient, wherein said formulation is substantially free of polyoxyethylene (20) sorbitan monolaurate. The invention also provides methods of using, a lyophilized or powdered form of, and processes for preparing the formulation.

The stable aqueous formulations of bovine granulocyte colony stimulating factor (“bG-CSF”) according to the invention contain a bG-CSF polypeptide or a variant thereof. As used herein, “bovine G-CSF polypeptide” (alternatively referred to as “bG-CSF polypeptide,” “bovine G-CSF,” or “bG-CSF”) and variants thereof shall include those polypeptides and proteins that have at least one biological activity of a CSF, bG-CSF analogs, bG-CSF mutants, altered glycosylated bG-CSF, PEG conjugated bG-CSF, bG-CSF isoforms, bG-CSF mimetics, bG-CSF fragments, hybrid bG-CSF proteins, fusion proteins, oligomers and multimers, homologues, glycosylation pattern variants, variants, splice variants, and muteins, thereof, regardless of the biological activity of same, and further regardless of the method of synthesis or manufacture thereof including, but not limited to, recombinant (whether produced from cDNA, genomic DNA, synthetic DNA or other form of nucleic acid), in vitro, in vivo, by microinjection of nucleic acid molecules, synthetic, transgenic, and gene activated methods. Additionally, the term bG-CSF polypeptide or a variant thereof encompasses bG-CSF polypeptides comprising one or more amino acid substitutions, additions or deletions. See U.S. Pat. No. 5,849,883 for examples of analogs of bovine G-CSF. The sequence of mature bG-CSF polypeptide is 174 amino acids in length is as follows (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

Furthermore, bG-CSF polypeptide with an initial methionine amino acid residue is as follows (SEQ ID NO: 2):

M T P L G P A R S L P Q S F L L K C L E Q V R K
I Q A D G A E L Q E R L C A A H K L C H P E E L
M L L R H S L G I P Q A P L S S C S S Q S L Q L
T S C L N Q L H G G L F L Y Q G L L Q A L A G I
S P E L A P T L D T L Q L D V T D F A T N I W L
Q M E D L G A A P A V Q P T Q G A M P T F T S A
F Q R R A G G V L V A S Q L H R F L E L A Y R G
L R Y L A E P

Substitutions in a wide variety of amino acid positions in bG-CSF have been described. Substitutions including but not limited to those that modulate pharmaceutical stability, increase agonist activity, increase protease resistance, convert the polypeptide into an antagonist, etc. are encompassed by the term bG-CSF polypeptide or a variant thereof.

The term bG-CSF polypeptide or a variant thereof also includes glycosylated bG-CSF, such as but not limited to polypeptides glycosylated at any amino acid position, N-linked glycosylated forms of the polypeptide, or O-linked glycosylated forms of the polypeptide. Variants containing single nucleotide changes are also considered as biologically active variants of bG-CSF polypeptide. Variants containing single nucleotide changes are also considered as biologically active variants of bG-CSF. In addition, splice variants are also included. The term bG-CSF polypeptide or a variant thereof also includes bG-CSF heterodimers, homodimers, heteromultimers, or homomultimers of any one or more bG-CSF or any other polypeptide, protein, carbohydrate, polymer, small molecule, linker, ligand, or other active molecule of any type, linked by chemical means or expressed as a fusion protein (see, for example, U.S. Pat. Nos. 6,261,550; 6,166,183; 6,204,247; 6,261,550; and 6,017,876), as well as polypeptide analogues containing, for example, specific deletions or other modifications yet maintain biological activity (see, for example, U.S. Pat. Nos. 6,261,550; 6,004,548; and 6,632,426). bG-CSF polypeptides and variants thereof are described, for example, in U.S. patent application Ser. No. 12/507,237 (now U.S. Patent Application Publication 2010/0035812), herein incorporated by reference. In some embodiments, one or more non-naturally encoded amino acids are incorporated in one or more of the following positions in bG-CSF: 8, 62, 69, 125, 133, 136, and any combination thereof (mature bG-CSF polypeptide or the corresponding amino acids in bG-CSF polypeptide with an initial methionine amino acid residue). In some embodiments, the non-naturally encoded amino acid para-acetylphenylalanine (pAF) is substituted for the naturally encoded amino acid at the one of the following positions: S8, S62, L69, G125, T133, A136, and any combination thereof (mature bG-CSF polypeptide or the corresponding amino acids in bG-CSF polypeptide with an initial methionine amino acid residue).

In one embodiment, the bG-CSF polypeptide or a variant thereof is bG-CSF-S8pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position S8.

In one embodiment, the bG-CSF polypeptide or a variant thereof is bG-CSF-S62pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position S62.

In one embodiment, the bG-CSF polypeptide or a variant thereof is bG-CSF-L69pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position L69.

In one embodiment, the bG-CSF polypeptide or a variant thereof is bG-CSF-G125pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position G125.

In one embodiment, the bG-CSF polypeptide or a variant thereof is bG-CSF-T133pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position T133.

In one embodiment, the bG-CSF polypeptide or a variant thereof is bG-CSF-A136pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position A136.

The formulation of the present invention can include a bG-CSF polypeptide or a variant thereof that is linked to a linker, a polymer, or a biologically active molecule. The linkers, polymers, and biologically active molecule are described in U.S. patent application Ser. No. 12/507,237 (now U.S. Patent Application Publication 2010/0035812). The term “linkage” or “linker” is used herein to refer to groups or bonds that normally are formed as the result of a chemical reaction and typically are covalent linkages. Hydrolytically stable linkages means that the linkages are substantially stable in water and do not react with water at useful pH values, including, but not limited to, under physiological conditions for an extended period of time, perhaps even indefinitely. Hydrolytically unstable or degradable linkages mean that the linkages are degradable in water or in aqueous solutions, including, for example, blood. Enzymatically unstable or degradable linkages mean that the linkage can be degraded by one or more enzymes. As understood in the art, PEG and related polymers may include degradable linkages in the polymer backbone or in the linker group between the polymer backbone and one or more of the terminal functional groups of the polymer molecule. For example, ester linkages formed by the reaction of PEG carboxylic acids or activated PEG carboxylic acids with alcohol groups on a biologically active agent generally hydrolyze under physiological conditions to release the agent. Other hydrolytically degradable linkages include, but are not limited to, carbonate linkages; imine linkages resulted from reaction of an amine and an aldehyde; phosphate ester linkages formed by reacting an alcohol with a phosphate group; hydrazone linkages which are reaction product of a hydrazide and an aldehyde; acetal linkages that are the reaction product of an aldehyde and an alcohol; orthoester linkages that are the reaction product of a formate and an alcohol; peptide linkages formed by an amine group, including but not limited to, at an end of a polymer such as PEG, and a carboxyl group of a peptide; and oligonucleotide linkages formed by a phosphoramidite group, including but not limited to, at the end of a polymer, and a 5′ hydroxyl group of an oligonucleotide.

In some embodiments, the bG-CSF polypeptide or a variant thereof is linked to a water soluble polymer. As used herein, the term “water soluble polymer” refers to any polymer that is soluble in aqueous solvents. Linkage of water soluble polymers to a bG-CSF polypeptide or a variant thereof can result in changes including, but not limited to, increased or modulated serum half-life, increased or modulated therapeutic half-life relative to the unmodified form, modulated immunogenicity, modulated physical association characteristics such as aggregation and multimer formation, altered receptor binding, altered binding to one or more binding partners, and altered receptor dimerization or multimerization. The water soluble polymer may or may not have its own biological activity, and may be utilized as a linker for attaching bG-CSF to other substances, including but not limited to one or more bG-CSF polypeptides or variants thereof, or one or more biologically active molecules. Suitable polymers include, but are not limited to, polyethylene glycol, polyethylene glycol propionaldehyde, mono C1-C10 alkoxy or aryloxy derivatives thereof (described in U.S. Pat. No. 5,252,714), monomethoxy-polyethylene glycol, polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polypropylene oxide/ethylene oxide copolymer, polyoxyethylated polyol, heparin, heparin fragments, polysaccharides, oligosaccharides, glycans, cellulose and cellulose derivatives, including but not limited to methylcellulose and carboxymethyl cellulose, starch and starch derivatives, polypeptides, polyalkylene glycol and derivatives thereof, copolymers of polyalkylene glycols and derivatives thereof, polyvinyl ethyl ethers, and alpha-beta-poly[(2-hydroxyethyl)-DL-aspartamide, and the like, or mixtures thereof. Examples of such water soluble polymers include, but are not limited to, polyethylene glycol and serum albumin. WO 03/074087 and WO 03/074088 describe the conjugation of proteins or small molecules to hydroxyalkyl starch (HAS). Examples of hydroxylalkyl starches, include but are not limited to, hydroxyethyl starch. Conjugates of hydroxyalkyl starch and another molecule, for example, may comprise a covalent linkage between terminal aldehyde groups of the HAS and reactive groups of the other molecule.

In some embodiments, the water soluble polymer is a poly(ethylene glycol) moiety. In some embodiments, the poly(ethylene glycol) moiety has a molecular weight of between about 0.1 kDa and about 100 kDa. In another embodiment, the water soluble polymer has a molecular weight of between about 0.1 kDa to about 50 kDa. In some embodiments, the water soluble polymer has a molecular weight of between about 10 kDa to about 30 kDa. In another embodiment, the water soluble polymer has a molecular weight of between about 15 kDa to about 25 kDa. In yet another embodiment, the water soluble polymer has a molecular weight of about 20 kDa. A person skilled in the art would understand that a water soluble polymer with a molecular weight of “about 20 kDa” includes variability in the molecular weight of approximately 15% (i.e., about 17 kDa to about 23 kDa) based on the specification and polydispersion of the moiety.

In one embodiment, the bG-CSF polypeptide or a variant thereof is bG-CSF-S8pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position S8 and is linked to a poly(ethylene glycol) moiety. For example, if the poly(ethylene glycol) moiety had a molecular weight of about 20 kDa, the bG-CSF polypeptide or a variant thereof in this embodiment could be identified as “bG-CSF-S8pAF-20K PEG”, indicating that a 20 kDa poly(ethylene glycol) moiety is linked to the pAF substitution made at position S8.

In one embodiment, the bG-CSF polypeptide or a variant thereof is bG-CSF-S62pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position S62 and is linked to a poly(ethylene glycol) moiety. For example, if the poly(ethylene glycol) moiety had a molecular weight of about 20 kDa, the bG-CSF polypeptide or a variant thereof in this embodiment could be identified as “bG-CSF-S62pAF-20K PEG”, indicating that a 20 kDa poly(ethylene glycol) moiety is linked to the pAF substitution made at position S62.

In one embodiment, the bG-CSF polypeptide or a variant thereof is bG-CSF-L69pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position L69 and is linked to a poly(ethylene glycol) moiety. For example, if the poly(ethylene glycol) moiety had a molecular weight of about 20 kDa, the bG-CSF polypeptide or a variant thereof in this embodiment could be identified as “bG-CSF-L69pAF-20K PEG”, indicating that a 20 kDa poly(ethylene glycol) moiety is linked to the pAF substitution made at position L69.

In one embodiment, the bG-CSF polypeptide or a variant thereof is bG-CSF-G125pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position G125 and is linked to a poly(ethylene glycol) moiety. For example, if the poly(ethylene glycol) moiety had a molecular weight of about 20 kDa, the bG-CSF polypeptide or a variant thereof in this embodiment could be identified as “bG-CSF-G125pAF-20K PEG”, indicating that a 20 kDa poly(ethylene glycol) moiety is linked to the pAF substitution made at position G125.

In one embodiment, the bG-CSF polypeptide or a variant thereof is bG-CSF-T133pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position T133 and is linked to a poly(ethylene glycol) moiety. For example, if the poly(ethylene glycol) moiety had a molecular weight of about 20 kDa, the bG-CSF polypeptide or a variant thereof in this embodiment could be identified as “bG-CSF-T133pAF-20K PEG”, indicating that a 20 kDa poly(ethylene glycol) moiety is linked to the pAF substitution made at position T133.

In one embodiment, the bG-CSF polypeptide or a variant thereof is bG-CSF-A136pAF, which has a sequence of (SEQ ID NO: 1):

T P L G P A R S L P Q S F L L K C L E Q V R K I
Q A D G A E L Q E R L C A A H K L C H P E E L M
L L R H S L G I P Q A P L S S C S S Q S L Q L T
S C L N Q L H G G L F L Y Q G L L Q A L A G I S
P E L A P T L D T L Q L D V T D F A T N I W L Q
M E D L G A A P A V Q P T Q G A M P T F T S A F
Q R R A G G V L V A S Q L H R F L E L A Y R G L
R Y L A E P

in which a single para-acetylphenylalanine (pAF) substitution is made at position A136 and is linked to a poly(ethylene glycol) moiety. For example, if the poly(ethylene glycol) moiety had a molecular weight of about 20 kDa, the bG-CSF polypeptide or a variant thereof in this embodiment could be identified as “bG-CSF-A136pAF-20K PEG”, indicating that a 20 kDa poly(ethylene glycol) moiety is linked to the pAF substitution made at position A136.

As used herein, the terms “stability” and “stable” in the context of a formulation comprising a bG-CSF polypeptide or a variant thereof refer to the thermal and chemical unfolding, aggregation, degradation, denaturation, fragmentation, or destabilization of the bG-CSF polypeptide or variant thereof under given manufacture, preparation, transportation and storage conditions. The “stable” formulations of the invention maintain structural integrity, which results in a retention of biological activity, desirably more than 80%, 85%, 90%, 95%, 98%, 99%, or 99.5% under given manufacture, preparation, transportation and storage conditions. The stability of the formulations can be assessed by degrees of aggregation, depegylation, degradation, denaturation, or fragmentation by methods known to those skilled in the art and described further herein.

As used herein, the term “aqueous” in the context of a formulation comprising a bG-CSF polypeptide or a variant thereof refers to water, one or more water-soluble organic solvents, or a mixture thereof. The term “organic solvent” is used herein in its conventional sense to refer to a liquid organic compound, typically a monomeric organic material in the form of a liquid, preferably a relatively non-viscous liquid, the molecular structure of which contains hydrogen atoms, carbon atoms, and optionally other atoms as well, and which is capable of dissolving solids gases or liquids.

The pharmaceutical formulations of the invention may comprise a pharmaceutically acceptable carrier. Pharmaceutically acceptable carriers are determined in part by the particular composition being administered, as well as by the particular method used to administer the composition (see, e.g., Remington's Pharmaceutical Sciences, 17th ed. 1985). Suitable pharmaceutically acceptable carriers include but are not limited to buffer substances and excipients, such as those containing saline, buffered saline, dextrose, water, glycerol, ethanol, and/or combinations thereof. Suitable carriers can be buffer substances containing succinate, phosphate, borate, HEPES, citrate, histidine, imidazole, acetate, bicarbonate, and other organic acids. Suitable carriers can be excipients containing polyhydric sugar alcohols, amino acids such as arginine, lysine, glycine, glutamine, asparagine, histidine, alanine, ornithine, leucine, phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Citrate, histidine, maleate, succinate, phosphate, or a combination thereof can be used according to the invention as buffer substances. In some embodiments, citrate or succinate is used as a buffer substance in the stable aqueous formulations. In some embodiments, the buffer substance has a molarity between about 10 mM and about 50 mM. In one embodiment, the buffer substance has a molarity of about 30 mM. The buffer substances can either be present in the form of the corresponding free acid or in the form of the alkali, alkaline-earth or ammonium salts. The formulation can in addition contain further common pharmaceutical auxiliary substances. The sequence of addition of the various auxiliary substances or of the active substance during the production of the liquid pharmaceutical formulations is largely independent of the stabilizing effect in storage found according to the invention and is at the discretion of the person skilled in the art.

Sodium chloride, trehalose, sorbitol, arginine, or a combination thereof can be used as excipients according to the invention. In one embodiment, the excipient is arginine. In some embodiments, arginine has a molarity between about 100 mM to about 500 mM. In other embodiments, arginine has a molarity of about 200 to about 300 mM. In some embodiments, arginine has a molarity of about 250 mM.

Traditionally, pharmaceutical formulations of proteins include surfactants. The inclusion of surfactants may protect proteins at potentially destabilizing interfaces, against surfaces encountered during processing, and against the alteration of their thermodynamic conformational stability. Surfactants are well known in the art, for example polysorbate surfactants. One example of a polysorbate surfactant is polyoxyethylene (20) sorbitan monolaurate, also known by the brand name Tween 20®. However, studies of a bG-CSF formulation containing trace levels of polyoxyethylene (20) sorbitan monolaurate indicate that aggregates increase up to 3.2% (as measured by size exclusion chromatography (SEC)) after 5 days of incubation at 25° C. Thus, the formulations of the present invention are substantially free of a surfactant, a polysorbate surfactant, and/or polyoxyethylene (20) sorbitan monolaurate. As used herein, the term “substantially free” of a surfactant, a polysorbate surfactant, and/or polyoxyethylene (20) sorbitan monolaurate refers to a formulation containing less than 0.033%, less than 0.001%, less than 0.0005%, less than 0.0003%, or less than 0.0001% of the surfactant, polysorbate surfactant, and/or polyoxyethylene (20) sorbitan monolaurate. The formulations of the present invention are substantially free of surfactant, polysorbate surfactant, and/or polyoxyethylene (20) sorbitan monolaurate in order to achieve a stable formulation with desirable properties, such as minimal product aggregation and minimal destabilization, and, where applicable, reduced depegylation.

The term “biologically active molecule” as used herein means any substance which can affect any physical or biochemical properties of a biological system, pathway, molecule, or interaction relating to an organism, including but not limited to, viruses, bacteria, bacteriophage, transposon, prion, insects, fungi, plants, animals, and humans. In particular, as used herein, biologically active molecules include, but are not limited to, any substance intended for diagnosis, cure, mitigation, treatment, or prevention of disease in humans or other animals, or to otherwise enhance physical or mental well-being of humans or animals. Examples of biologically active molecules include, but are not limited to, peptides, proteins, enzymes, small molecule drugs, vaccines, immunogens, hard drugs, soft drugs, carbohydrates, inorganic atoms or molecules, dyes, lipids, nucleosides, radionuclides, oligonucleotides, toxoids, toxins, prokaryotic and eukaryotic cells, viruses, polysaccharides, nucleic acids and portions thereof obtained or derived from viruses, bacteria, insects, animals or any other cell or cell type, liposomes, microparticles and micelles.

The pharmaceutical formulations of the present invention include those that also optionally contain one or more other active ingredients, in addition to a bG-CSF polypeptide or a variant thereof. As used herein, the term “active ingredient” or “therapeutic ingredient” refers to a therapeutically active compound, as well as any prodrugs thereof and pharmaceutically acceptable salts, hydrates and solvates of the compound and the prodrugs. Other active ingredients may be combined with a bG-CSF polypeptide or a variant thereof and may be either administered separately or in the same pharmaceutical formulation. The amount of other active ingredients to be given may be readily determined by one skilled in the art based upon therapy with bG-CSF.

The pharmaceutical formulations of the present invention include those that also optionally contain one or more other inactive ingredients, in addition to a bG-CSF polypeptide or a variant thereof. As used herein, the term “inactive ingredient” refers to a therapeutically inactive compound, as well as any prodrugs thereof and pharmaceutically acceptable salts, hydrates and solvates of the compound and the prodrugs. Other inactive ingredients may be combined with a bG-CSF polypeptide or a variant thereof and may be either administered separately or in the same pharmaceutical formulation. The amount of other inactive ingredients to be given may be readily determined by one skilled in the art based upon therapy with bG-CSF.

The amount of the bG-CSF polypeptide or a variant thereof in the stable aqueous formulations is adequate to achieve a therapeutic effect. As used herein, the term “therapeutically effective amount” refers to an amount which gives the desired benefit to an animal and includes both treatment and prophylactic administration. The amount will vary from one individual to another and will depend upon a number of factors, including the overall physical condition of the patient and the underlying cause of the condition to be treated. The amount of bG-CSF polypeptide or variant thereof used for therapy gives an acceptable rate of change and maintains desired response at a beneficial level. A therapeutically effective amount of the present compositions may be readily ascertained by one of ordinary skill in the art using publicly available materials and procedures. For example, the amount of the bG-CSF polypeptide or variant thereof can be present in the formulation in an amount of between about 0.5 and about 12 grams/liter, preferably about 5 grams/liter.

According to the present invention, the stable aqueous formulations of a bG-CSF polypeptide or variant thereof can be formulated at various pH values. In some embodiments, the stable aqueous formulation can have a pH value of between about 5.7 to about 6.6. In one embodiment, the stable aqueous formulation has a pH of between about 6.0 to about 6.3. The desired pH value of the formulation is adjusted by adding bases such as alkali hydroxides, alkaline-earth hydroxides or ammonium hydroxide. Sodium hydroxide is preferably used for pH adjustment. The adjustment of the desired pH value can in principle be achieved by adding basic solutions. In general, salts of strong bases with weak acids can be used, such as sodium acetate, sodium citrate, di-sodium or di-potassium hydrogen phosphate or sodium carbonate. If the pharmaceutical solution of auxiliary substance has a basic pH value, it is adjusted by titration with an acid until the desired pH range of 4-5 or 7-8 is reached. Physiologically tolerated inorganic or organic acids come into consideration as acids such as for example hydrochloric acid, phosphoric acid, acetic acid, citric acid, or conventional solutions of substances which have an acidic pH value. In this respect, some example substances are salts of strong acids with weak bases such as e.g., sodium dihydrogen phosphate or potassium dihydrogen phosphate.

As demonstrated in the examples below, bG-CSF formulations of the present invention show desirably low aggregate concentrations of bG-CSF polypeptide or a variant thereof at stressed storage conditions and at accelerated storage conditions. As used herein, stressed storage conditions are evaluated after formulation samples are incubated at 25° C. for 5 days. As used herein, accelerated storage conditions are evaluated after formulation samples are incubated at 40° C. for 1 day. Storage conditions can also be evaluated at other various temperatures and durations for the purposes of the present invention. For example, storage conditions could be evaluated after formulation samples are incubated at 25° C. for 28 days or after formulation samples are incubated at 40° C. for 3 days.

Aggregate concentration of bG-CSF polypeptide or a variant thereof are analyzed following stressed storage conditions and accelerated storage conditions. In some embodiments, bG-CSF formulations of the present invention have an aggregate concentration of bG-CSF polypeptide or a variant thereof of less than about 2.1% (weight/weight percentage) at stressed storage conditions. In other embodiments, bG-CSF formulations of the present invention have an aggregate concentration of bG-CSF polypeptide or a variant thereof of less than about 1.5% (weight/weight percentage) at stressed storage conditions. In some embodiments, bG-CSF formulations of the present invention have an aggregate concentration of bG-CSF polypeptide or a variant thereof of less than about 1.5% (weight/weight percentage) at accelerated storage conditions. In other embodiments, bG-CSF formulations of the present invention have an aggregate concentration of bG-CSF polypeptide or a variant thereof of less than about 1.5% (weight/weight percentage) at stressed storage conditions.

In addition, forced agitation studies or freeze-thaw studies can be utilized to assess stability properties of a formulation of the present invention. For example, a forced agitation study could consist of mixing a formulation sample in a glass beaker at a set speed, such as 60 rpm, using a magnetic stirrer. The agitation could occur for a determined period of time, such as two hours, in order to determine the characteristics of the formulation sample. A freeze-thaw cycle could consist of freezing a formulation sample for 1 hour at about −75° C. and thawing at room temperature for approximately 1 hour until no ice was observed.

Moreover, as demonstrated in the examples below, bG-CSF formulations of the present invention can show desirable destabilization and/or depegylation properties of bG-CSF polypeptide or a variant thereof at stressed storage conditions and at accelerated storage conditions. As used herein, the term “depegylation” can refer to the stability of the attachment of pegylated moieties bound to a bG-CSF polypeptide or a variant thereof, i.e. whether such pegylated moieties remain bound to the polypeptide over time, for example during storage in an aqueous solution, or whether they tend to detach, for example as a result of ester bond hydrolysis.

In some embodiments, the stable aqueous formulations of a bG-CSF polypeptide or variant thereof can be formulated using citrate as a buffer substance and arginine as an excipient. In one embodiment, the aqueous formulation can be prepared using citric acid monohydrate (Fisher, C/6200/60 or equivalent) as the buffer substance and L-Arginine (Sigma, A8094 or equivalent) as the excipient. The aqueous formulation can be prepared by adding 1.6±0.1 grams of citric acid monohydrate and 10.9±0.1 grams of L-arginine to 200 mL of high quality water. Thereafter, the pH can be adjusted to 6.0±0.1 using hydrochloric acid and the mixture can be diluted to 250 mL using high quality water. The resultant formulation can comprise 30 mM citrate, 250 mM arginine, and a bG-CSF polypeptide or variant thereof at a pH of 6.0.

The aqueous preparations according to the invention can be used to produce lyophilisates by conventional lyophilization or powders. The preparations according to the invention are obtained again by dissolving the lyophilisates in water or other aqueous solutions. The term “lyophilization,” also known as freeze-drying, is a commonly employed technique for presenting proteins which serves to remove water from the protein preparation of interest. Lyophilization is a process by which the material to be dried is first frozen and then the ice or frozen solvent is removed by sublimation in a vacuum environment. An excipient may be included in pre-lyophilized formulations to enhance stability during the freeze-drying process and/or to improve stability of the lyophilized product upon storage. For example, see Pikal, M. Biopharm. 3(9)26-30 (1990) and Arakawa et al. Pharm. Res. 8(3):285-291 (1991).

The spray drying of pharmaceutical ingredients is also known to those of ordinary skill in the art. For example, see Broadhead, J. et al., “The Spray Drying of Pharmaceuticals,” in Drug Dev. Ind. Pharm, 18 (11 & 12), 1169-1206 (1992). In addition to small molecule pharmaceuticals, a variety of biological materials have been spray dried including: enzymes, sera, plasma, micro-organisms and yeasts. Spray drying is a useful technique because it can convert a liquid pharmaceutical preparation into a fine, dustless or agglomerated powder in a one-step process. The basic technique comprises the following four steps: a) atomization of the feed solution into a spray; b) spray-air contact; c) drying of the spray; and d) separation of the dried product from the drying air. For example, U.S. Pat. Nos. 6,235,710 and 6,001,800 describe the preparation of recombinant erythropoietin by spray drying.

Methods of using a formulation containing a bG-CSF polypeptide or a variant thereof are also encompassed by the present invention. bG-CSF has a variety of biological activities including but not limited to binding to its receptor, causing dimerization of its receptor, stimulation of neutrophil production, and stimulating cell proliferation and differentiation. Examples of some of the biological activities of granulocyte colony stimulating factor and bG-CSF are described above and in U.S. Pat. Nos. 6,676,947; 6,579,525; 6,531,121; 6,521,245; 6,489,293; 6,368,854; 6,316,254; 6,268,336; 6,239,109; 6,165,283; 5,986,047; 5,830,851; 5,043,156; and 5,773,569. The formulations containing b-GCSF polypeptide or a variant thereof of the invention are useful for treating or preventing a wide range of disorders. “Preventing” refers to reducing the likelihood that the recipient will incur or develop any of the pathological conditions described herein and includes prophylactic administration. The term “preventing” is particularly applicable to a patient that is susceptible to the particular patholical condition. “Treating” refers to mediating a disease or condition and preventing, reversing the clinical effects of the disease, or mitigating, its further progression or ameliorate the symptoms associated with the disease or condition.

Administration of G-CSF products results in white blood cell formation. Thus, administration of a formulation containing bG-CSF polypeptide or a variant thereof of the present invention may be useful to prevent infection in animals that are at risk of infection. A formulation containing bG-CSF polypeptide or a variant thereof of the present invention may be administered to animals that have an infection. Infections that may be treated with a formulation containing bG-CSF polypeptide or a variant thereof of the invention include, but are not limited to, mastitis and shipping fever. A formulation containing a bG-CSF polypeptide or a variant thereof of the present invention could be administered to an animal, for example, between two weeks and one day before giving birth and optionally an additional administration could be given on the day of giving birth or up to one week after giving birth. In some embodiments, the animal that is administered the formulation containing a bG-CSF polypeptide or a variant thereof of the present invention is a cow, and giving birth is referred to as “calving.” In one embodiment, a formulation containing bG-CSF polypeptide or a variant thereof of the present invention may be administered to periparturient cows for the prevention of mastitis.

According to the invention, a formulation containing bG-CSF polypeptide or a variant thereof may be administered by any conventional route suitable for proteins or peptides, including, but not limited to, parenterally, e.g. injections including, but not limited to, subcutaneously or intravenously or any other form of injections or infusions. Formulations containing bG-CSF polypeptide or a variant thereof can be administered by a number of routes including, but not limited to oral, intravenous, intraperitoneal, intramuscular, transdermal, subcutaneous, topical, sublingual, intravascular, intramammary, or rectal means. Formulations containing bG-CSF polypeptide or a variant thereof can also be administered via liposomes. Such administration routes and appropriate formulations are generally known to those of skill in the art. Formulations containing bG-CSF polypeptide or a variant thereof, alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations containing bG-CSF polypeptide or a variant thereof suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations containing bG-CSF polypeptide or a variant thereof can be presented in unit-dose or multi-dose sealed containers, such as ampules and vials. The formulations containing bG-CSF polypeptide or a variant thereof can also be presented in syringes, such as prefilled syringes.

Parenteral administration and intravenous administration are possible methods of administration of the formulations of the present invention. In particular, the routes of administration already in use for natural amino acid homologue therapeutics (including but not limited to, those typically used for EPO, GH, G-CSF, GM-CSF, IFNs, interleukins, antibodies, FGFs, and/or any other pharmaceutically delivered protein), along with formulations in current use, provide possible routes of administration and formulations containing bG-CSF polypeptide or a variant thereof of the invention.

In some embodiments, the formulations of the present invention containing bG-CSF polypeptide or a variant thereof in an amount between about 0.5 and about 12 grams/liter. The dose administered to an animal, in the context of the present invention, is sufficient to have a beneficial therapeutic response in the animal over time, or other appropriate activity, depending on the application. The dose is determined by the efficacy of the particular vector, or formulation, and the activity, stability or serum half-life of the unnatural amino acid polypeptide employed and the condition of the animal, as well as the body weight or surface area of the animal to be treated. The size of the dose is also determined by the existence, nature, and extent of any adverse side-effects that accompany the administration of a particular vector, formulation, or the like in a particular animal.

The dose administered to an animal in the context of the present invention should be sufficient to cause a beneficial response in the subject over time. Generally, the total pharmaceutically effective amount of the bG-CSF polypeptide or a variant thereof of the present invention administered parenterally per dose is in the range of about 0.01 μg/kg/day to about 100 μg/kg, or about 0.05 mg/kg to about 1 mg/kg, of animal body weight, although this is subject to therapeutic discretion. Alternatively, the pharmaceutically effective amount of the bG-CSF polypeptide or a variant thereof of the present invention administered parenterally per dose is about 1 mg to about 25 mg, or about 5 mg to about 20 mg. For example, the pharmaceutically effective amount of the bG-CSF polypeptide or a variant thereof of the present invention administered parenterally per dose can be about 14 mg. The frequency of dosing is also subject to therapeutic discretion.

The pharmaceutically effective amount of the bG-CSF polypeptide or a variant thereof may be administered to animals as a single dose or as part of a multi-dose schedule. For example, the bG-CSF polypeptide or a variant thereof may be administered in a multi-dose schedule wherein the schedule is at least a two dose regimen. In one embodiment, the multi-dose schedule is a two dose regimen.

In one embodiment, the multi-dose schedule comprises a first dose administered to an animal about 1 days to about 14 days before the animal gives birth and the second dose is administered to the animal about 4 days prior to about 7 days after the animal gives birth. In another embodiment, the multi-dose schedule comprises a first dose administered to an animal about 7 days before the animal gives birth and the second dose is administered to the animal on the day the animal gives birth.

The following embodiments are also contemplated:
1. A stable aqueous formulation comprising a bG-CSF polypeptide or a variant thereof, a buffer substance, and an excipient, wherein said formulation is substantially free of polyoxyethylene (20) sorbitan monolaurate.
2. The formulation of clause 1 wherein the bG-CSF polypeptide or the variant thereof is linked to a linker, a polymer, or a biologically active molecule.
3. The formulation of clause 1 or clause 2 wherein the bG-CSF polypeptide or the variant thereof is linked to a water soluble polymer.
4. The formulation of clause 3 wherein the water soluble polymer comprises a poly(ethylene glycol) moiety.
5. The formulation of clause 3 or clause 4 wherein the water soluble polymer has a molecular weight of between about 0.1 kDa and about 100 kDa.
6. The formulation of any one of clauses 3 to 5 wherein the water soluble polymer has a molecular weight of between about 0.1 kDa and about 50 kDa.
7. The formulation of any one of clauses 3 to 6 wherein the water soluble polymer has a molecular weight of about 20 kDa.
8. The formulation of any one of clauses 1 to 7 wherein the bG-CSF polypeptide or variant thereof is bG-CSF-T133pAF-20K PEG.
9. The formulation of any one of clauses 1 to 8 wherein bG-CSF polypeptide or variant thereof is present in an amount of between about 0.5 and about 12 grams/liter.
10. The formulation of any one of clauses 1 to 9 wherein bG-CSF polypeptide or variant thereof is present in an amount of about 5 grams/liter.
11. The formulation of any one of clauses 1 to 10 wherein the buffer substance is citrate, histidine, maleate, succinate, phosphate, or a combination thereof.
12. The formulation of any one of clauses 1 to 11 wherein the buffer substance is citrate or succinate.
13. The formulation of any one of clauses 1 to 12 wherein the buffer substance is citrate.
14. The formulation of any one of clauses 1 to 12 wherein the buffer substance is succinate.
15. The formulation of any one of clauses 1 to 14 wherein the buffer substance has a molarity between about 10 mM and about 50 mM.
16. The formulation of any one of clauses 1 to 15 wherein the buffer substance has a molarity of about 30 mM.
17. The formulation of any one of clauses 1 to 16 wherein the excipient is sodium chloride, trehalose, sorbitol, arginine, or a combination thereof.
18. The formulation of any one of clauses 1 to 17 wherein the excipient is arginine.
19. The formulation of clause 18 wherein arginine has a molarity of between about 100 mM to about 500 mM.
20. The formulation of clause 18 or clause 19 wherein arginine has a molarity of about 200 to about 300 mM.
21. The formulation of any one of clauses 18 to 20 wherein arginine has a molarity of about 250 mM.
22. The formulation of any one of clauses 1 to 21 wherein the formulation has a pH of between about 5.7 to about 6.6.
23. The formulation of any one of clauses 1 to 22 wherein the formulation has a pH of between about 6.0 to about 6.3.
24. The formulation of any one of clauses 1 to 23 wherein the formulation has an average aggregate concentration of bG-CSF polypeptide or variant thereof of less than about 2.1% wt/wt % after a five day incubation period at stressed storage conditions.
25. The formulation of any one of clauses 1 to 24 wherein the formulation has an average aggregate concentration of bG-CSF polypeptide or variant thereof of less than about 1.5% wt/wt % after a one day incubation period at accelerated storage conditions.
26. The formulation of any one of clauses 1 to 25 optionally including one or more other therapeutic ingredients.
27. A lyophilisate or powder of the formulation of any one of clauses 1 to 26.
28. An aqueous solution produced by dissolving the lyophilisate or powder of clause 27 in water.
29. A process for preparing the formulation of any one of clauses 1 to 26 comprising forming a stable aqueous solution comprising bG-CSF polypeptide or a variant thereof, a buffer substance, and an excipient, wherein said formulation is substantially free of polyoxyethylene (20) sorbitan monolaurate.
30. A method of treating an animal having a disorder modulated by bG-CSF comprising administering to said animal a therapeutically effective amount of the formulation of any one of clauses 1 to 26.
31. The method of clause 30 wherein said disorder is an infection.
32. The method of clause 31 wherein said infection is mastitis and wherein said animal is a periparturient cow.
33. A stable aqueous formulation comprising a bG-CSF polypeptide or a variant thereof, a citrate or succinate buffer, arginine, and optionally a counter ion for arginine.
34. The formulation of clause 33 wherein the formulation is substantially free of a polysorbate surfactant.
35. The formulation of clause 33 or clause 34 wherein the bG-CSF polypeptide or the variant thereof is linked to a linker, a polymer, or a biologically active molecule.
36. The formulation of any one of clauses 33 to 35 wherein the bG-CSF polypeptide or the variant thereof is linked to a water soluble polymer.
37. The formulation of clause 36 wherein the water soluble polymer comprises a poly(ethylene glycol) moiety.
38. The formulation of clause 36 or clause 37 wherein the water soluble polymer has a molecular weight of between about 0.1 kDa and about 100 kDa.
39. The formulation of any one of clauses 36 to 38 wherein the water soluble polymer has a molecular weight of between about 0.1 kDa and about 50 kDa.
40. The formulation of any one of clauses 36 to 39 wherein the water soluble polymer has a molecular weight of about 20 kDa.
41. The formulation of any one of clauses 34 to 40 wherein said polysorbate surfactant is a polyoxyethylene derivative of sodium monolaurate.
42. The formulation of any one of clauses 34 to 41 wherein said polysorbate surfactant is polyoxyethylene (20) sorbitan monolaurate.
43. The formulation of any one of clauses 33 to 42 wherein the bG-CSF polypeptide or variant thereof is bG-CSF-T133pAF-20K PEG.
44. The formulation of clause 43 wherein bG-CSF-T133pAF-20K PEG is present in an amount of between about 0.5 and about 12 grams/liter, the citrate buffer has a molarity of about 30 mM, arginine has a molarity of about 250 mM, and wherein the formulation has a pH value of about 6.0.
45. The formulation of clause 43 or clause 44 wherein the formulation has an average aggregate concentration of bG-CSF-T133pAF-20K PEG of less than about 1.6% wt/wt % after a 28-day incubation period at 25° C.
46. The formulation of any one of clauses 43 to 45 wherein the formulation has an average aggregate concentration of bG-CSF-T133pAF-20K PEG of less than about 2.8% wt/wt % after a 3-day incubation period at 40° C.
47. The formulation of any one of clauses 43 to 46 wherein the formulation has an average aggregate concentration of bG-CSF-T133pAF-20K PEG of about 1.6% wt/wt % or less after a forced agitation study.
48. The formulation of any one of clauses 43 to 47 wherein the formulation has an average aggregate concentration of bG-CSF-T133pAF-20K PEG of less than about 1.6% wt/wt % after five freeze-thaw cycles.
49. The formulation of any one of clauses 33 to 48 wherein the counter ion for arginine is chloride or sulfate.
50. The formulation of any one of clauses 33 to 49 optionally including one or more other therapeutic ingredients.
51. A lyophilisate or powder of the formulation of any one of clauses 33 to 50.
52. An aqueous solution produced by dissolving the lyophilisate or powder of clause 51 in water.
53. A process for preparing the formulation of any one of clauses 33 to 50 comprising forming a stable aqueous solution comprising a bG-CSF polypeptide or variant thereof, a citrate buffer, arginine, and optionally a counter ion for arginine.
54. The process of clause 53 wherein the formulation is substantially free of a polysorbate surfactant.
55. The process of clause 53 or clause 54 wherein the bG-CSF polypeptide or variant thereof is bG-CSF-T133pAF-20K PEG.
56. A method of treating an animal having a disorder modulated by bG-CSF comprising administering to said animal a therapeutically effective amount of the formulation of any one of clauses 33 to 50.
57. The method of clause 56 wherein said disorder is an infection.
58. The method of clause 57 wherein said infection is mastitis and wherein said animal is a periparturient cow.
59. A stable aqueous formulation consisting essentially of a bG-CSF polypeptide or a variant thereof, a citrate or succinate buffer, arginine, and optionally a counter ion for arginine.
60. The formulation of clause 59 wherein the formulation is substantially free of a surfactant.
61. The formulation of clause 59 or clause 60 wherein the bG-CSF polypeptide or the variant thereof is linked to a linker, a polymer, or a biologically active molecule.
62. The formulation of any one of clauses 59 to 61 wherein the bG-CSF polypeptide or the variant thereof is linked to a water soluble polymer.
63. The formulation of clause 62 wherein the water soluble polymer comprises a poly(ethylene glycol) moiety.
64. The formulation of clause 62 or clause 63 wherein the water soluble polymer has a molecular weight of between about 0.1 kDa and about 100 kDa.
65. The formulation of any one of clauses 62 to 64 wherein the water soluble polymer has a molecular weight of between about 0.1 kDa and about 50 kDa.
66. The formulation of any one of clauses 62 to 65 wherein the water soluble polymer has a molecular weight of about 20 kDa.
67. The formulation of any one of clauses 60 to 66 wherein said surfactant is a polysorbate surfactant.
68. The formulation of any one of clauses 60 to 67 wherein said surfactant is a polyoxyethylene derivative of sodium monolaurate.
69. The formulation of any one of clauses 60 to 68 wherein said surfactant is polyoxyethylene (20) sorbitan monolaurate.
70. The formulation of any one of clauses 59 to 69 wherein the bG-CSF polypeptide or variant thereof is bG-CSF-T133pAF-20K PEG.
71. The formulation of clause 70 wherein bG-CSF-T133pAF-20K PEG is present in an amount of between about 0.5 and about 12 grams/liter, the citrate buffer has a molarity of about 30 mM, arginine has a molarity of about 250 mM, and wherein the formulation has a pH value of about 6.0.
72. The formulation of clause 70 or clause 71 wherein the formulation has an average aggregate concentration of bG-CSF-T133pAF-20K PEG of less than about 1.6% wt/wt % after a 28-day incubation period at 25° C.
73. The formulation of any one of clauses 70 to 72 wherein the formulation has an average aggregate concentration of bG-CSF-T133pAF-20K PEG of less than about 2.8% wt/wt % after a 3-day incubation period at 40° C.
74. The formulation of any one of clauses 70 to 73 wherein the formulation has an average aggregate concentration of bG-CSF-T133pAF-20K PEG of about 1.6% wt/wt % or less after a forced agitation study.
75. The formulation of any one of clauses 70 to 74 wherein the formulation has an average aggregate concentration of bG-CSF-T133pAF-20K PEG of less than about 1.6% wt/wt % after five freeze-thaw cycles.
76. The formulation of any one of clauses 59 to 75 wherein the counter ion for arginine is chloride or sulfate.
77. The formulation of any one of clauses 59 to 76 optionally including one or more other therapeutic ingredients.
78. A lyophilisate or powder of the formulation of any one of clauses 59 to 77.
79. An aqueous solution produced by dissolving the lyophilisate or powder of clause 78 in water.
80. A process for preparing the formulation of any one of clauses 59 to 77 comprising forming a stable aqueous solution consisting essentially of a bG-CSF polypeptide or variant thereof, a citrate buffer, arginine, and optionally a counter ion for arginine.
81. The process of clause 80 wherein the formulation is substantially free of a surfactant.
82. The process of clause 80 or clause 81 wherein the bG-CSF polypeptide or variant thereof is bG-CSF-T133pAF-20K PEG.
83. A method of treating an animal having a disorder modulated by bG-CSF comprising administering to said animal a therapeutically effective amount of the formulation of any one of clauses 59 to 77.
84. The method of clause 83 wherein said disorder is an infection.
85. The method of clause 84 wherein said infection is mastitis and wherein said animal is a periparturient cow.
86. A stable aqueous formulation consisting essentially of bG-CSF-T133pAF-20K PEG, a citrate buffer wherein the citrate buffer has a molarity of about 30 mM, arginine wherein arginine has a molarity of about 250 mM, and optionally a counter ion for arginine.

### EXAMPLE 1

### Buffer and Excipient Screening Study

bGCSF-T133-20K PEG formulations without polyoxyethylene (20) sorbitan monolaurate in the background can be screened to assess product stability using multiple buffers and excipients (sodium chloride, trehalose, and arginine). The target pH for all dialysis buffers is pH 6.0. For comparison, a formulation containing 10 mM phosphate, 180 mM mannitol, and 60 mM trehalose at pH 6.0 can be prepared. The formulations can be evaluated for effects on protein aggregation and depegylation in the presence and absence of oxygen.

The samples can be prepared by dialyzing 1 mL of bGCSF-T133-20K PEG at 2-8° C. into each formulation. Protein concentration of the dialyzed samples can be determined before normalizing the protein concentration to 5 mg/mL. After dialysis and concentration normalization, approximately 3×1 mL of the post-dialyzed and diluted pool can be filled into 5 mL glass vials. One set of samples can be tested to provide initial conditions. A second set can be stored at 25° C./60% RH for 5 days before testing. The third set of samples can be degassed in the lyophilization chamber, closed under an inert atmosphere (nitrogen), and then stored at 25° C./60% RH for 5 days before testing. If the level of aggregate as measured by SEC after 5 days is ≤2.0%, both the degassed and non-degassed samples can be incubated at 40° C. for one day.

After five days of incubation, protein concentration of each sample can be measured. Table 1 shows protein concentrations.

TABLE 1
Protein Concentrations from the Buffer and Excipient Screening Study

| | | | Post 5-Day | Post 5-Day |
|---|---|---|---|---|
| | | Post 5-Day | at 25° C. | at 26° C. |
| | | at 2-8° C. | Concentration | Concentration |
| Sample | | Concentration | (mg/mL) | (mg/mL) |
| No. | Sample Description | (mg/mL) | UN-DEGAS | DEGAS |
| 1 | 10 mM Citrate, 0.1M Arginine | 5.76 | 5.29 | 5.93 |
| 2 | 10 mM Citrate, 0.15M NaCl | 6.36 | 6.71 | 5.45 |
| 3 | 10 mM Citrate, 0.3M Trehalose | 6.71 | 5.78 | 6.79 |
| 4 | 10 mM Histidine, 0.15M NaCl | 5.39 | 5.19 | 6.42 |
| 5 | 10 mM Histidine, 0.3M Trehalose | 5.30 | 5.08 | 5.34 |
| 6 | 10 mM Histidine, 0.1M Arginine | 5.55 | 5.06 | 6.27 |
| 7 | 10 mM Maleate, 0.15M NaCl | 5.60 | 5.40 | 5.73 |
| 8 | 10 mM Maleate, 0.3M Trehalose | 4.41 | 4.01 | 4.33 |
| 9 | 10 mM Maleate, 0.1M Arginine | 5.09 | 5.41 | 5.39 |
| 10 | 10 mM Succinate, 0.15M NaCl | 5.94 | 5.68 | 5.83 |
| 11 | 10 mM Succinate, 0.3M Trehalose | 5.57 | 5.79 | 5.66 |
| 12 | 10 mM Succinate, 0.1M Arginine | 4.96 | 4.82 | 4.78 |
| 13 | 10 mM Phosphate, 0.15M NaCl | 5.20 | 5.18 | 5.03 |
| 14 | 10 mM Phosphate, 0.3M Trehalose | 5.13 | 5.30 | 5.24 |
| 15 | 10 mM Phosphate, 0.1M Arginine | 5.22 | 5.04 | 5.12 |
| 16 | 10 mM Phosphate, | 5.28 | 5.22 | 6.21 |
| | 180 mM Mannitol, | | | |
| | 60 mM Trehalose | | | |

Table 2 shows the pH results for each sample.

TABLE 2
pH Results from the Buffer and Excipient Screening Study

| | Dialysis | Post | 5-Day | 5-Day | 5-Day 25 C. |
|---|---|---|---|---|---|
| Buffer Condition | Buffer pH | Dialysis pH | 2-8 C. | 25 C. | Degassed pH |
| 10 mM Citrate, 0.1M Arginine | 6.49 | 6.44 | 5.48 | 6.57 | 6.53 |
| 10 mM Citrate, 0.15M NaCl | 6.51 | 6.47 | 6.52 | 5.55 | 6.61 |
| 10 mM Citrate, 0.3M Trehalose | 6.11 | 6.07 | 6.20 | 6.16 | 6.18 |
| 10 mM Histidine, 0.15M NaCl | 5.86 | 5.83 | 5.91 | 5.92 | 5.95 |
| 10 mM Histidine, 0.3M Trehalose | 5.78 | 5.81 | 5.92 | 5.96 | 5.95 |
| 10 mM Histidine, 0.1M Arginine | 6.22 | 6.23 | 6.30 | 6.32 | 6.29 |
| 10 mM Maleate, 0.15M NaCl | 6.22 | 6.22 | 6.28 | 6.32 | 6.29 |
| 10 mM Maleate, 0.3M Trehalose | 6.06 | 6.03 | 6.11 | 6.13 | 6.12 |
| 10 mM Maleate, 0.1M Arginine | 6.24 | 6.23 | 6.34 | 6.34 | 6.32 |
| 10 mM Succinate, 0.15M NaCl | 6.14 | 6.23 | 6.19 | 5.25 | 6.31 |
| 10 mM Succinate, 0.3M Trehalose | 6.09 | 6.07 | 6.13 | 6.13 | 6.16 |
| 10 mM Succinate, 0.1M Arginine | 6.14 | 6.14 | 6.30 | 5.34 | 6.34 |
| 10 mM Phosphate, 0.15M NaCl | 6.29 | 6.25 | 5.30 | 6.33 | 6.37 |
| 10 mM Phosphate, 0.3M Trehalose | 5.97 | 5.98 | 6.03 | 6.10 | 6.13 |
| 10 mM Phosphate, 0.1M Arginine | 6.40 | 6.38 | 6.41 | 6.47 | 6.47 |
| 10 mM Phosphate, | 6.09 | 6.04 | 6.14 | 6.11 | 6.12 |
| 180 mM Mannitol, | | | | | |
| 60 mM Trehalose | | | | | |

Protein aggregation and depegylation levels in each formulation can be analyzed by SEC. Table 3 shows the SEC results and indicates that the aggregation and depegylation levels were similar across all samples.

TABLE 3
SEC Results from the Buffer and Excipient Screening Study (Post 5 Days Incubations)

| | | | | | | | 5-Day Incubation at | | |
|---|---|---|---|---|---|---|---|---|---|
| | Control Samples (2-8° C.) | | | 5-Day Incubation at 25° C. | | | 25° C. (DEGAS) | | |
| | | Avg % | | | Avg % | | | Avg % | |
| | Avg % | PEG- | Avg % | Avg % | PEG- | Avg % | Avg % | PEG- | Avg % |
| Sample | Aggregate | bGCSF | bGCSF | Aggregate | bGCSF | bGCSF | Aggregate | bGCSF | bGCSF |
| Pre-dialyzed | 0.7 | 98.5 | 0.7 | N/A | | | | | |
| bGCSF-T133- | | | | | | | | | |
| 20K PEG | | | | | | | | | |
| NBJ0801-04-04 | | | | | | | | | |
| 10 mM Citrate, | 1.2 | 98.3 | 0.5 | 1.6 | 98.0 | 0.5 | 1.6 | 98.0 | 0.4 |
| 0.15 M NaCl | | | | | | | | | |
| 10 mM Citrate, | 1.3 | 98.3 | 0.4 | 1.5 | 98.1 | 0.4 | 1.7 | 97.8 | 0.4 |
| 0.3 M Trehalose | | | | | | | | | |
| 10 mM Citrate, | 1.3 | 98.2 | 0.4 | 1.4 | 98.1 | 0.5 | 1.5 | 98.0 | 0.5 |
| 0.1 M Arginine | | | | | | | | | |
| 10 mM Histidine, | 1.3 | 98.1 | 0.6 | 1.5 | 97.8 | 0.7 | 1.6 | 97.8 | 0.7 |
| 0.15 M NaCl | | | | | | | | | |
| 10 mM Histidine, | 1.3 | 98.2 | 0.5 | 1.7 | 97.8 | 0.5 | 1.9 | 97.6 | 0.5 |
| 0.3 M Trehalose | | | | | | | | | |
| 10 mM Histidine, | 1.3 | 98.1 | 0.6 | 1.3 | 98.1 | 0.7 | 1.5 | 97.8 | 0.6 |
| 0.1 M Arginine | | | | | | | | | |
| 10 mM Maleate, | 1.4 | 98.1 | 0.5 | 1.6 | 97.8 | 0.6 | 1.7 | 97.7 | 0.6 |
| 0.15 M NaCl | | | | | | | | | |
| 10 mM Maleate, | 1.3 | 98.2 | 0.4 | 1.7 | 97.9 | 0.4 | 1.7 | 97.9 | 0.4 |
| 0.3 M Trehalose | | | | | | | | | |
| 10 mM Maleate, | 1.3 | 98.2 | 0.6 | 1.3 | 98.1 | 0.6 | 1.4 | 97.9 | 0.6 |
| 0.1 M Arginine | | | | | | | | | |
| 10 mM Succinate, | 1.4 | 98.1 | 0.5 | 1.6 | 97.8 | 0.6 | 1.9 | 97.5 | 0.5 |
| 0.15 M NaCl | | | | | | | | | |
| 10 mM Succinate, | 1.3 | 98.3 | 0.4 | 1.7 | 97.9 | 0.4 | 1.9 | 97.7 | 0.4 |
| 0.3 M Trehalose | | | | | | | | | |
| 10 mM Succinate, | 1.5 | 98.0 | 0.6 | 1.5 | 97.9 | 0.6 | 2.0 | 97.4 | 0.6 |
| 0.1 M Arginine | | | | | | | | | |
| 10 mM Phosphate, | 1.1 | 98.2 | 0.7 | 1.3 | 98.0 | 0.7 | 1.4 | 97.9 | 0.7 |
| 0.15 M NaCl | | | | | | | | | |
| 10 mM Phosphate, | 1.1 | 98.4 | 0.5 | 1.7 | 97.8 | 0.6 | 1.8 | 97.6 | 0.6 |
| 0.3 M Trehalose | | | | | | | | | |
| 10 mM Phosphate, | 1.1 | 98.2 | 0.6 | 1.3 | 98.1 | 0.7 | 1.5 | 97.8 | 0.7 |
| 0.1 M Arginine | | | | | | | | | |
| 10 mM Phosphate, | 1.2 | 98.3 | 0.5 | 2.0 | 97.5 | 0.5 | 2.1 | 97.4 | 0.5 |
| 0.18 M Mannitol, | | | | | | | | | |
| 0.06 M Trehalose | | | | | | | | | |

Table 4 shows the SEC results for samples incubated at 40° C. for one day. Comparison of the aggregate composition indicates that formulations containing arginine had the lowest product aggregation. Furthermore, the reference formulation 10 mM phosphate, 180 mM Mannitol, and 60 mM Trehalose pH 6 has the highest level of aggregation if compared to all the other formulations in the screening study.

TABLE 4
SEC Results from the Buffer and Excipient Screening Study (Post 1 Day Incubation)

| | | | | 1-Day Incubation at | | |
|---|---|---|---|---|---|---|
| | 1-Day Incubation at 40° C. | | | 40° C. (DEGAS) | | |
| | | Avg % | | | Avg % | |
| | Avg % | PEG- | Avg % | Avg % | PEG- | Avg % |
| Sample | Aggregate | bGCSF | bGCSF | Aggregate | bGCSF | bGCSF |
| 10 mM Citrate, | 6.5 | 93.3 | 0.1 | 7.2 | 92.7 | 0.1 |
| 0.15 M NaCl | | | | | | |
| 10 mM Citrate, | 4.1 | 95.8 | 0.1 | 4.3 | 95.6 | 0.1 |
| 0.3 M Trehalose | | | | | | |
| 10 mM Citrate, | 3.9 | 95.9 | 0.2 | 3.3 | 96.5 | 0.2 |
| 0.1 M Arginine | | | | | | |
| 10 mM Histidine, | 6.0 | 93.8 | 0.2 | 5.9 | 93.9 | 0.2 |
| 0.15 M NaCl | | | | | | |
| 10 mM Histidine, | 8.4 | 91.5 | 0.1 | 9.3 | 90.6 | 0.1 |
| 0.3 M Trehalose | | | | | | |
| 10 mM Histidine, | 3.0 | 96.7 | 0.3 | 2.9 | 96.8 | 0.3 |
| 0.1 M Arginine | | | | | | |
| 10 mM Maleate, | 5.9 | 93.9 | 0.2 | 6.0 | 93.9 | 0.1 |
| 0.15 M NaCl | | | | | | |
| 10 mM Maleate, | 7.3 | 92.7 | 0.1 | 7.4 | 92.5 | 0.1 |
| 0.3 M Trehalose | | | | | | |
| 10 mM Maleate, | 3.0 | 96.8 | 0.2 | 3.1 | 96.7 | 0.2 |
| 0.1 M Arginine | | | | | | |
| 10 mM Succinate, | 4.3 | 95.5 | 0.2 | 5.3 | 94.6 | 0.1 |
| 0.15 M NaCl | | | | | | |
| 10 mM Succinate, | 9.6 | 90.3 | 0.1 | 10.8 | 89.1 | 0.1 |
| 0.3 M Trehalose | | | | | | |
| 10 mM Succinate, | 2.1 | 97.6 | 0.3 | 2.6 | 97.2 | 0.2 |
| 0.1 M Arginine | | | | | | |
| 10 mM Phosphate, | 5.9 | 94.0 | 0.2 | 5.9 | 93.9 | 0.1 |
| 0.15 M NaCl | | | | | | |
| 10 mM Phosphate, | 16.0 | 84.0 | 0.0 | 18.2 | 81.8 | 0.0 |
| 0.3 M Trehalose | | | | | | |
| 10 mM Phosphate, | 4.5 | 95.2 | 0.2 | 3.8 | 96.0 | 0.2 |
| 0.1 M Arginine | | | | | | |
| 10 mM Phosphate, | 22.8 | 77.2 | 0.0 | 25.0 | 75.0 | 0.0 |
| 0.18 M Mannitol, | | | | | | |
| 0.06 M Trehalose | | | | | | |

Results from this screening study indicate that succinate, histidine, maleate, and citrate formulations without polyoxyethylene (20) sorbitan monolaurate all have negligible aggregate increase (less than 1% by SEC) after a five day incubation at 25° C. Also, no difference exists in protein stability between the degassed and non-degassed samples. Furthermore, SEC results from stressed samples at 40° C. for one day show that formulations containing 0.1 M arginine have less aggregation compared with formulations containing sodium chloride and trehalose excipients.

### EXAMPLE 2

### Effect of Polyoxyethylene (20) Sorbitan Monolaurate on bG-CSF Formulations

The effect of polyoxyethylene (20) sorbitan monolaurate on aggregation can be evaluated to determine the impact on future formulations for agitation studies. The samples can be prepared by dialyzing 4 mL of bGCSF-T133-20K PEG at 2-8° C. into 10 mM Phosphate and 150 mM NaCl at pH 6.0. Following dialysis, the dialyzed pool can be spiked with a 1% polyoxyethylene (20) sorbitan monolaurate stock solution and then can be diluted with 10 mM Phosphate and 150 mM NaCl at pH 6.0 to a final protein concentration of 5 mg/mL. Samples from each formulation can be divided into 2×1 mL aliquots filled in 1 mL glass vials to form two sets of samples. One set can be stored at 2-8° C. and tested at initial conditions; a second set can be can be stored at 40° C. for one day.

Table 5 shows the SEC integration data and indicates that the aggregation level increases with increasing polyoxyethylene (20) sorbitan monolaurate concentration. SEC analysis of the samples indicates that bGCSF-T133-20K PEG aggregation increases with polyoxyethylene (20) sorbitan monolaurate concentration. As a result, polyoxyethylene (20) sorbitan monolaurate can be excluded from future formulation testing for bGCSF-T133-20K PEG.

TABLE 5
SEC Results from the Polyoxyethylene (20) Sorbitan
Monolaurate Study (Post 1 Day Incubation)

| | Initial | | | 1-Day Incubation at 40° C. | | |
|---|---|---|---|---|---|---|
| | | Avg % | | | Avg % | |
| | Avg % | PEG- | Avg % | Avg % | PEG- | Avg % |
| Buffer | Aggregate | bGCSF | bGCSF | Aggregate | bGCSF | bGCSF |
| Pre-dialyzed Pool | 0.7 | 99.0 | 0.3 | N/A | | |
| NBJ0801-04-04 | | | | | | |
| 10 mM Phosphate, | 0.8 | 98.2 | 1.0 | 2.6 | 96.6 | 0.8 |
| 150 mM NaCl | | | | | | |
| 10 mM Phosphate, | 0.8 | 98.1 | 1.1 | 3.7 | 95.6 | 0.7 |
| 150 mM NaCl, | | | | | | |
| 0.0033% Tween-20 | | | | | | |
| 10 mM Phosphate, | 0.9 | 98.0 | 1.1 | 9.2 | 90.1 | 0.7 |
| 150 mM NaCl, | | | | | | |
| 0.05% Tween-20 | | | | | | |

### EXAMPLE 3

### Box-Behnken Response Surface Design (DOE #1)

The effect of various arginine concentrations along with other key historical formulation parameters can be tested to evaluate the main effects as well as their interactions. The experimental design can be a Box-Behnken response surface where each numeric factor is varied at the low, center, and high level. Furthermore, the buffer type can be a categorical factor. The parameter combination can be duplicated for citrate and succinate, each with three centerpoints. The pH can be set at 6.0 for all conditions. A control condition comprising 10 mM Phosphate, 150 mM NaCl, and 0.0033% polyoxyethylene (20) sorbitan monolaurate at pH 6 can be included for comparison with historical results.

All dialysis buffers can be prepared at pH 6.0±0.1. PEG-bGCSF can be dialyzed into 18 buffer conditions that represent all the buffer conditions of the DOE #1 study. The protein recovery across the dialysis step can be generally ≥78% and, thus, is consistent within the dialysis sample set. Following dialysis, the protein concentration of dialyzed pool can be adjusted with the dialysis buffer to the target value shown in the Box-Behnken response surface design. This could result in 24 formulation combinations plus three centerpoints in citrate and three centerpoints in succinate. Each formulation can be divided into 3×1 mL aliquots filled in 1 mL glass vials to form three sets of samples: one set can be tested as initial conditions and then stored at 2-8° C., a second can be stored at 25° C. for two weeks, and the third set can be stored at 40° C. for one day.

Changes in product concentration can be analyzed to assess product stability. Table 6 shows the product concentration of samples before and after incubation. Samples at 10 mM Citrate, 300 mM Arginine (8 mg/mL) and 10 mM Succinate, 300 mM Arginine (8 mg/mL) have the highest increase (0.5-0.6 mg/mL) whereas the difference is less for all other samples.

TABLE 6
Summary of Protein Concentration from the DOE #1
Study (Initial and 1-day at 40° C.)

| | Protein Concentration (mg/mL) | | |
|---|---|---|---|
| | | 40° C. | |
| Sample | Initial | for 1 Day | Difference |
| 10 mM Citrate, 100 mM Arginine (6 mg/mL) | 4.89 | 5.16 | 0.27 |
| 10 mM Citrate, 300 mM Arginine (2 mg/mL) | 1.99 | 2.05 | 0.06 |
| 10 mM Citrate, 300 mM Arginine (8 mg/mL) | 7.60 | 8.19 | 0.59 |
| 10 mM Citrate, 500 mM Arginine (5 mg/mL) | 4.97 | 4.89 | −0.08 |
| 30 mM Citrate, 100 mM Arginine (2 mg/mL) | 1.99 | 2.01 | 0.02 |
| 30 mM Citrate, 100 mM Arginine (8 mg/mL) | 8.23 | 8.14 | −0.09 |
| 30 mM Citrate, 300 mM Arginine (5 mg/mL) Vial A | 5.03 | 4.86 | −0.17 |
| 30 mM Citrate, 300 mM Arginine (5 mg/mL) Vial B | 5.07 | 4.93 | −0.14 |
| 30 mM Citrate, 300 mM Arginine (5 mg/mL) Vial C | 5.08 | 5.01 | −0.07 |
| 30 mM Citrate, 500 mM Arginine (2 mg/mL) | 2.00 | 2.01 | 0.01 |
| 30 mM Citrate, 500 mM Arginine (8 mg/mL) | 8.05 | 7.95 | −0.10 |
| 50 mM Citrate, 100 mM Arginine (5 mg/mL) | 6.07 | 5.01 | −0.06 |
| 50 mM Citrate, 300 mM Arginine (2 mg/mL) | 1.99 | 2.00 | 0.01 |
| 50 mM Citrate, 300 mM Arginine (8 mg/mL) | 8.17 | 8.17 | 0.00 |
| 50 mM Citrate, 500 mM Arginine (6 mg/mL) | 4.89 | 4.93 | 0.04 |
| 10 mM Succinate, 100 mM Arginine (5 mg/mL) | 6.26 | 5.08 | −0.19 |
| 10 mM Succinate, 300 mM Arginine (2 mg/mL) | 1.94 | 1.88 | −0.06 |
| 10 mM Succinate, 300 mM Arginine (6 mg/mL) | 8.21 | 8.72 | 0.61 |
| 10 mM Succinate, 500 mM Arginine (5 mg/mL) | 6.03 | 4.91 | −0.12 |
| 30 mM Succinate, 100 mM Arginine (2 mg/mL) | 2.04 | 1.75 | −0.29 |
| 30 mM Succinate, 100 mM Arginine (6 mg/mL) | 7.97 | 7.79 | −0.16 |
| 30 mM Succinate, 300 mM Arginine (5 mg/mL) Vial A | 4.92 | 4.77 | −0.15 |
| 30 mM Succinate, 300 mM Arginine (6 mg/mL) Vial B | 4.87 | 4.85 | −0.02 |
| 30 mM Succinate, 300 mM Arginine (5 mg/mL) Vial C | 4.97 | 4.80 | −0.17 |
| 30 mM Succinate, 500 mM Arginine (2 mg/mL) | 1.90 | 4.88 | −0.04 |
| 30 mM Succinate, 500 mM Arginine (8 mg/mL) | 7.81 | 7.69 | −0.12 |
| 50 mM Succinate, 100 mM Arginine (6 mg/mL) | 4.80 | 4.94 | 0.14 |
| 50 mM Succinate, 300 mM Arginine (2 mg/mL) | 1.88 | 1.64 | −0.02 |
| 50 mM Succinate, 300 mM Arginine (6 mg/mL) | 7.86 | 7.76 | −0.12 |
| 50 mM Succinate, 600 mM Arginine (5 mg/mL) | 4.91 | 4.92 | 0.00 |
| 10 mM Phosphate, 160 mM NaCl, 0.0033% | 6.05 | 5.02 | −0.02 |
| Tween-20 (w/v) (6 mg/mL)4 | | | |

Changes in pH can be analyzed to assess pH stability of the samples. All sample pH can be within the range of 6.0-6.3. Table 7 shows the pH values and the difference from the time zero. Sample pH is stable for the entire duration of the DOE #1 study.

TABLE 7
Summary of pH from the DOE #1
Study (Initial and 1-day at 40° C.)

| | pH | | |
|---|---|---|---|
| | | 40° C. | |
| Sample | Initial | for 1 Day | Difference |
| 10 mM Citrate, 100 mM Arginine (6 mg/mL) | 6.14 | 6.18 | 0.02 |
| 10 mM Citrate, 300 mM Arginine (2 mg/mL) | 6.10 | 6.12 | 0.02 |
| 10 mM Citrate, 300 mM Arginine (8 mg/mL) | 6.08 | 6.10 | 0.02 |
| 10 mM Citrate, 500 mM Arginine (5 mg/mL) | 6.25 | 6.27 | 0.02 |
| 30 mM Citrate, 100 mM Arginine (2 mg/mL) | 6.16 | 6.19 | 0.04 |
| 30 mM Citrate, 100 mM Arginine (8 mg/mL) | 6.13 | 6.19 | 0.06 |
| 30 mM Citrate, 300 mM Arginine (5 mg/mL) Vial A | 6.19 | 6.21 | 0.02 |
| 30 mM Citrate, 300 mM Arginine (5 mg/mL) Vial B | 6.31 | 6.21 | −0.10 |
| 30 mM Citrate, 300 mM Arginine (5 mg/mL) Vial C | 6.24 | 6.21 | −0.03 |
| 30 mM Citrate, 500 mM Arginine (2 mg/mL) | 6.16 | 6.16 | 0.00 |
| 30 mM Citrate, 500 mM Arginine (8 mg/mL) | 6.12 | 6.15 | 0.03 |
| 50 mM Citrate, 100 mM Arginine (5 mg/mL) | 6.28 | 6.22 | −0.06 |
| 50 mM Citrate, 300 mM Arginine (2 mg/mL) | 6.19 | 6.18 | −0.01 |
| 50 mM Citrate, 300 mM Arginine (8 mg/mL) | 6.19 | 6.16 | −0.03 |
| 50 mM Citrate, 500 mM Arginine (6 mg/mL) | 6.19 | 6.15 | −0.04 |
| 10 mM Succinate, 100 mM Arginine (5 mg/mL) | 6.11 | 6.18 | 0.05 |
| 10 mM Succinate, 300 mM Arginine (2 mg/mL) | 6.06 | 6.08 | 0.06 |
| 10 mM Succinate, 300 mM Arginine (6 mg/mL) | 6.04 | 6.05 | 0.01 |
| 10 mM Succinate, 500 mM Arginine (5 mg/mL) | 6.17 | 6.17 | 0.00 |
| 30 mM Succinate, 100 mM Arginine (2 mg/mL) | 6.25 | 6.23 | −0.02 |
| 30 mM Succinate, 100 mM Arginine (6 mg/mL) | 6.25 | 6.23 | −0.02 |
| 30 mM Succinate, 300 mM Arginine (5 mg/mL) Vial A | 6.31 | 6.31 | 0.00 |
| 30 mM Succinate, 300 mM Arginine (6 mg/mL) Vial B | 6.30 | 6.30 | 0.00 |
| 30 mM Succinate, 300 mM Arginine (5 mg/mL) Vial C | 6.30 | 6.29 | −0.01 |
| 30 mM Succinate, 500 mM Arginine (2 mg/mL) | 6.19 | 6.19 | 0.00 |
| 30 mM Succinate, 500 mM Arginine (8 mg/mL) | 6.19 | 6.18 | −0.01 |
| 50 mM Succinate, 100 mM Arginine (6 mg/mL) | 6.18 | 6.17 | −0.01 |
| 50 mM Succinate, 300 mM Arginine (2 mg/mL) | 6.22 | 6.19 | −0.03 |
| 50 mM Succinate, 300 mM Arginine (6 mg/mL) | 6.21 | 6.21 | 0.00 |
| 50 mM Succinate, 600 mM Arginine (5 mg/mL) | 6.22 | 6.21 | −0.01 |
| 10 mM Phosphate, 160 mM NaCl, 0.0033% | 6.11 | 6.10 | −0.01 |
| Tween-20 (w/v) (6 mg/mL)4 | | | |

Changes in SEC aggregate, monomer, and depegylation levels can be analyzed to assess protein stability. Tables 8, 9 and 10 show the compositions of aggregation, monomer, and depegylation in each sample composition, respectively.

TABLE 8
SEC Aggregate Results from DOE #1 Study

| | % Aggregate for Citrate | | | % Aggregate for Succinate | | |
|---|---|---|---|---|---|---|
| | | 40° C. | | | 40° C. | |
| Sample | Initial | for 1 Day | Difference | Initial | for 1 Day | Difference |
| Pre-dialyzed Pool | 1.1 | N/A | | 0.07 | N/A | |
| NBJ0801-04-04 | | | | | | |
| for (Buffer) | | | | | | |
| 10 mM Buffer, | 1.1 | 1.5 | 0.4 | 1.3 | 2.9 | 1.6 |
| 100 mM Arginine | | | | | | |
| (5 mg/mL) | | | | | | |
| 10 mM Buffer, | 1.0 | 1.0 | 0.0 | 1.5 | 1.4 | −0.1 |
| 300 mM Arginine | | | | | | |
| (2 mg/mL) | | | | | | |
| 10 mM Buffer, | 1.0 | 1.2 | 0.2 | 1.2 | 1.4 | 0.3 |
| 300 mM Arginine | | | | | | |
| (8 mg/mL) | | | | | | |
| 10 mM Buffer, | 1.1 | 1.3 | 0.2 | 1.1 | 1.4 | 0.2 |
| 500 mM Arginine | | | | | | |
| (5 mg/mL) | | | | | | |
| 30 mM Buffer, | 1.2 | 1.3 | 0.0 | 1.6 | 1.7 | 0.1 |
| 100 mM Arginine | | | | | | |
| (2 mg/mL) | | | | | | |
| 30 mM Buffer, | 1.1 | 1.5 | 0.4 | 1.2 | 2.6 | 1.3 |
| 100 mM Arginine | | | | | | |
| (8 mg/mL) | | | | | | |
| 30 mM Buffer, | 1.1 | 1.1 | 0.0 | 1.4 | 1.4 | 0.0 |
| 300 mM Arginine | | | | | | |
| (5 mg/mL) Vial A | | | | | | |
| 30 mM Buffer, | 1.0 | 1.2 | 0.2 | 1.2 | 1.5 | 0.3 |
| 300 mM Arginine | | | | | | |
| (5 mg/mL) Vial B | | | | | | |
| 30 mM Buffer, | 1.0 | 1.1 | 0.1 | 1.2 | 1.4 | 0.2 |
| 300 mM Arginine | | | | | | |
| (5 mg/mL) Vial C | | | | | | |
| 30 mM Buffer, | 1.1 | 1.2 | 0.1 | 1.9 | 1.4 | −0.5 |
| 500 mM Arginine | | | | | | |
| (2 mg/mL) | | | | | | |
| 30 mM Buffer, | 1.1 | 1.3 | 0.2 | 1.3 | 1.4 | 0.1 |
| 500 mM Arginine | | | | | | |
| (8 mg/mL) | | | | | | |
| 50 mM Buffer, | 1.3 | 1.5 | 0.2 | 1.5 | 2.7 | 1.3 |
| 100 mM Arginine | | | | | | |
| (5 mg/mL) | | | | | | |
| 50 mM Buffer, | 1.2 | 1.1 | −0.1 | 1.6 | 1.4 | −0.2 |
| 300 mM Arginine | | | | | | |
| (2 mg/mL) | | | | | | |
| 50 mM Buffer, | 1.1 | 1.4 | 0.3 | 1.3 | 1.8 | 0.5 |
| 300 mM Arginine | | | | | | |
| (8 mg/mL) | | | | | | |
| 50 mM Buffer, | 1.4 | 1.2 | −0.2 | 1.4 | 1.4 | 0.0 |
| 500 mM Arginine | | | | | | |
| (5 mg/mL) | | | | | | |

| | | | |
|---|---|---|---|
| 10 mM Phosphate, | Initial = 0.8 | 40° C. for | Difference = 2.8 |
| 150 mM NaCl, | | 1 Day = 3.7 | |
| 0.05% Tween-20 | | | |
| (5 mg/mL) | | | |

TABLE 9
SEC Monomer Results from DOE #1 Study

| | % Monomer for Citrate | | | % Monomer for Succinate | | |
|---|---|---|---|---|---|---|
| | | 40° C. | | | 40° C. | |
| Sample | Initial | for 1 Day | Difference | Initial | for 1 Day | Difference |
| Pre-dialyzed Pool | 98.7 | N/A | | 99.0 | N/A | |
| NBJ0801-04-04 | | | | | | |
| for (Buffer) | | | | | | |
| 10 mM Buffer, | 98.8 | 98.4 | −0.4 | 97.9 | 96.6 | −1.4 |
| 100 mM Arginine | | | | | | |
| (5 mg/mL) | | | | | | |
| 10 mM Buffer, | 98.8 | 98.7 | −0.1 | 97.9 | 98.0 | 0.2 |
| 300 mM Arginine | | | | | | |
| (2 mg/mL) | | | | | | |
| 10 mM Buffer, | 98.9 | 98.6 | −0.3 | 97.8 | 97.7 | −0.1 |
| 300 mM Arginine | | | | | | |
| (8 mg/mL) | | | | | | |
| 10 mM Buffer, | 98.7 | 98.4 | −0.3 | 97.9 | 97.8 | −0.1 |
| 500 mM Arginine | | | | | | |
| (5 mg/mL) | | | | | | |
| 30 mM Buffer, | 98.6 | 98.5 | −0.1 | 98.0 | 97.9 | −0.1 |
| 100 mM Arginine | | | | | | |
| (2 mg/mL) | | | | | | |
| 30 mM Buffer, | 98.7 | 98.3 | −0.4 | 98.1 | 96.9 | −1.2 |
| 100 mM Arginine | | | | | | |
| (8 mg/mL) | | | | | | |
| 30 mM Buffer, | 98.7 | 98.6 | −0.1 | 98.0 | 98.1 | 0.1 |
| 300 mM Arginine | | | | | | |
| (5 mg/mL) Vial A | | | | | | |
| 30 mM Buffer, | 98.8 | 98.5 | −0.2 | 98.2 | 98.0 | −0.2 |
| 300 mM Arginine | | | | | | |
| (5 mg/mL) Vial B | | | | | | |
| 30 mM Buffer, | 98.8 | 98.6 | −0.2 | 98.2 | 98.1 | −0.1 |
| 300 mM Arginine | | | | | | |
| (5 mg/mL) Vial C | | | | | | |
| 30 mM Buffer, | 98.7 | 98.5 | −0.2 | 97.6 | 98.1 | 0.4 |
| 500 mM Arginine | | | | | | |
| (2 mg/mL) | | | | | | |
| 30 mM Buffer, | 98.7 | 98.4 | −0.3 | 98.0 | 98.0 | 0.0 |
| 500 mM Arginine | | | | | | |
| (8 mg/mL) | | | | | | |
| 50 mM Buffer, | 98.5 | 98.3 | −0.2 | 98.1 | 96.9 | −1.2 |
| 100 mM Arginine | | | | | | |
| (5 mg/mL) | | | | | | |
| 50 mM Buffer, | 98.6 | 98.6 | 0.0 | 97.9 | 98.2 | 0.2 |
| 300 mM Arginine | | | | | | |
| (2 mg/mL) | | | | | | |
| 50 mM Buffer, | 98.7 | 98.3 | −0.3 | 98.1 | 97.7 | −0.4 |
| 300 mM Arginine | | | | | | |
| (8 mg/mL) | | | | | | |
| 50 mM Buffer, | 98.4 | 98.5 | 0.1 | 98.0 | 98.0 | 0.0 |
| 500 mM Arginine | | | | | | |
| (5 mg/mL) | | | | | | |

| | | | |
|---|---|---|---|
| 10 mM Phosphate, | Initial = 98.1 | 40° C. for | Difference = −2.5 |
| 150 mM NaCl, | | 1 Day = 95.6 | |
| 0.0033% Tween-20 | | | |
| (5 mg/mL) | | | |

TABLE 10
SEC Depegylation Results from DOE #1 Study

| | % Depegylation for Citrate | | | % Depegylation for Succinate | | |
|---|---|---|---|---|---|---|
| | | 40° C. | | | 40° C. | |
| Sample | Initial | for 1 Day | Difference | Initial | for 1 Day | Difference |
| Pre-dialyzed Pool | 0.2 | N/A | | 0.3 | N/A | |
| NBJ0801-04-04 | | | | | | |
| for (Buffer) | | | | | | |
| 10 mM Buffer, | 0.2 | 0.1 | 0.0 | 0.8 | 0.6 | −0.3 |
| 100 mM Arginine | | | | | | |
| (5 mg/mL) | | | | | | |
| 10 mM Buffer, | 0.2 | 0.3 | 0.1 | 0.6 | 0.6 | 0.0 |
| 300 mM Arginine | | | | | | |
| (2 mg/mL) | | | | | | |
| 10 mM Buffer, | 0.2 | 0.3 | 0.1 | 1.0 | 0.8 | −0.2 |
| 300 mM Arginine | | | | | | |
| (8 mg/mL) | | | | | | |
| 10 mM Buffer, | 0.2 | 0.3 | 0.1 | 1.0 | 0.8 | −0.1 |
| 500 mM Arginine | | | | | | |
| (5 mg/mL) | | | | | | |
| 30 mM Buffer, | 0.2 | 0.2 | 0.0 | 0.4 | 0.4 | 0.0 |
| 100 mM Arginine | | | | | | |
| (2 mg/mL) | | | | | | |
| 30 mM Buffer, | 0.2 | 0.2 | 0.0 | 0.6 | 0.5 | −0.1 |
| 100 mM Arginine | | | | | | |
| (8 mg/mL) | | | | | | |
| 30 mM Buffer, | 0.2 | 0.3 | 0.1 | 0.6 | 0.5 | −0.1 |
| 300 mM Arginine | | | | | | |
| (5 mg/mL) Vial A | | | | | | |
| 30 mM Buffer, | 0.2 | 0.3 | 0.1 | 0.6 | 0.5 | −0.1 |
| 300 mM Arginine | | | | | | |
| (5 mg/mL) Vial B | | | | | | |
| 30 mM Buffer, | 0.2 | 0.2 | 0.0 | 0.6 | 0.5 | 0.0 |
| 300 mM Arginine | | | | | | |
| (5 mg/mL) Vial C | | | | | | |
| 30 mM Buffer, | 0.2 | 0.3 | 0.1 | 0.5 | 0.5 | 0.0 |
| 500 mM Arginine | | | | | | |
| (2 mg/mL) | | | | | | |
| 30 mM Buffer, | 0.2 | 0.3 | 0.1 | 0.7 | 0.7 | −0.1 |
| 500 mM Arginine | | | | | | |
| (8 mg/mL) | | | | | | |
| 50 mM Buffer, | 0.2 | 0.2 | 0.0 | 0.4 | 0.4 | −0.1 |
| 100 mM Arginine | | | | | | |
| (5 mg/mL) | | | | | | |
| 50 mM Buffer, | 0.2 | 0.3 | 0.1 | 0.4 | 0.4 | 0.0 |
| 300 mM Arginine | | | | | | |
| (2 mg/mL) | | | | | | |
| 50 mM Buffer, | 0.2 | 0.2 | 0.0 | 0.6 | 0.6 | −0.1 |
| 300 mM Arginine | | | | | | |
| (8 mg/mL) | | | | | | |
| 50 mM Buffer, | 0.2 | 0.3 | 0.1 | 0.6 | 0.6 | 0.0 |
| 500 mM Arginine | | | | | | |
| (5 mg/mL) | | | | | | |

| | | | |
|---|---|---|---|
| 10 mM Phosphate, | Initial = 1.1 | 40° C. for | Difference = −0.3 |
| 150 mM NaCl, | | 1 Day = 0.7 | |
| 0.0033% Tween-20 | | | |
| (5 mg/mL) | | | |

The SEC results indicate that the aggregate level in citrate samples is relatively unchanged. Succinate samples also have low aggregate levels except for samples with 100 mM arginine, suggesting that succinate-based buffers would require more than 100 mM arginine to maintain low protein aggregation. The control condition (10 mM Phosphate, 150 mM NaCl, and 0.0033% (w/v) polyoxyethylene (20) sorbitan monolaurate at pH 6 and at 5 mg/mL) have 3.7% aggregate after one day incubation at 40° C. (see Table 8).

Depegylation is another protein degradation pathway. Table 10 shows SEC results for depegylated product and indicates that the depegylation level in succinate samples is higher (0.4%- 0.8%) than those in citrate samples (≤0.3%). The depegylation level in the phosphate control is higher than all citrate formulation samples and is slightly higher than most succinate formulations.

Since SEC results for citrate samples incubated at 40° C. for one day have minimal aggregate, a subset of the DOE #1 samples can be incubated at 25° C. for 28 days. The following sample conditions can be analyzed by SEC:
1. 30 mM Citrate, 100 mM Arginine at 2 mg/mL
2. 30 mM Citrate, 500 mM Arginine at 2 mg/mL
3. 30 mM Citrate, 100 mM Arginine at 8 mg/mL
4. 30 mM Citrate, 500 mM Arginine at 8 mg/mL
5. 30 mM Citrate, 300 mM Arginine at 5 mg/mL

Table 11 shows the SEC results of the 28 day experiment. Moreover, analysis by RP-HPLC can be performed on the samples incubated at 28 days to ensure lack of product degradation. Table 12 shows these results.

TABLE 11
SEC Results from DOE #1 Study Incubated 28 days at 25° C.

| | 28-Day Incubation at 25° C. | | |
|---|---|---|---|
| | Avg % | Avg % | Avg % |
| Sample | Aggregate | PEG-bGCSF | bGCSF |
| 100 mM Arginine 2 mg/mL | 1.3 | 98.1 | 0.6 |
| 100 mM Arginine 8 mg/mL | 1.6 | 98.0 | 0.4 |
| 300 mM Arginine 5 mg/mL Vial A | 1.4 | 98.0 | 0.7 |
| 300 mM Arginine 5 mg/mL Vial B | 1.3 | 98.1 | 0.6 |
| 300 mM Arginine 5 mg/mL Vial C | 1.3 | 98.0 | 0.7 |
| 500 mM Arginine 2 mg/mL | 1.4 | 97.8 | 0.8 |
| 500 mM Arginine 6 mg/mL | 1.5 | 97.7 | 0.7 |

TABLE 12
RP-HPLC Results from DOE #1 Study Incubated 28 days at 25° C.

| | % Monomer | | |
|---|---|---|---|
| | | 26 Day at | |
| | Initial | 40° C. | Difference |
| 100 mM Arginine 2 mg/mL | 97.3 | 97.8 | 0.4 |
| 100 mM Arginine 8 mg/mL | 97.0 | 97.4 | 0.4 |
| 300 mM Arginine 5 mg/mL Vial A | 97.3 | 97.3 | 0.0 |
| 300 mM Arginine 5 mg/mL Vial B | 97.1 | 95.2 | −2.0 |
| 300 mM Arginine 5 mg/mL Vial C | 97.1 | 97.4 | 0.3 |
| 500 mM Arginine 2 mg/mL | 97.2 | 97.0 | −0.2 |
| 500 mM Arginine 6 mg/mL | 97.2 | 97.2 | 0.0 |

### EXAMPLE 4

### Counter Ion and Syringe Compatibility Evaluation

A comparison of chloride and sulfate as counter ions for arginine can be evaluated. The sample condition can be 30 mM citrate and 300 mM arginine at 5 mg/mL (pH 6). In addition, product compatibility in MONOJECT 3 mL polypropylene syringe for containing the drug product can be compared to 1 mL glass vials. The 30 mM citrate, 300 mM arginine pH 6 (Chloride) buffer can be prepared using sodium citrate and arginine-HCI, and the solution can be titrated with 6N HCI. The 30 mM citrate, 300 mM arginine pH 6 (Sulfate) buffer can be prepared using citric acid monohydrate, sodium citrate, and arginine base, and the solution can be titrated with concentrated sulfuric acid. bGCSFT133-20K PEG can be dialyzed into the two buffers. Samples can be analyzed by SEC at time zero. One set of samples can be placed in 1 mL glass lyophilization vial and a second in 3 mL syringes prior to incubation at 40° C. for up to 3 days.

Table 13 shows SEC results. The SEC results indicate that aggregate formation is two to three times higher in samples stored in syringes than in glass vials. Product depegylation remains the same as the time zero samples. For both counter ions, samples in glass vials have minimal change in aggregate after 3 days at 40° C. Chloride could be used in place of sulfate as a counter ion without impact on aggregate formation.

TABLE 13
SEC Results from the Counter Ion and Syringe Compatibility Evaluation

| | Initial | | | 1-Day Incubation at 40° C. | | | 2-Day Incubation at 40° C. | | | 3-Day Incubation at 40° C. | | |
|---|---|---|---|---|---|---|---|---|---|---|---|---|
| | % | % PEG- | % | % | % PEG- | % | % | % PEG- | % | % | % PEG- | % |
| Sample | Aggregate | bGCSF | bGCSF | Aggregate | bGCSF | bGCSF | Aggregate | bGCSF | bGCSF | Aggregate | bGCSF | bGCSF |
| Pre-dialyzed | 0.5 | 99.2 | 0.3 | N/A | | | | | | | | |
| Pool | | | | | | | | | | | | |
| Chloride | 1.2 | 98.5 | 0.3 | 1.2 | 98.5 | 0.4 | 1.2 | 98.3 | 0.4 | 1.2 | 98.2 | 0.7 |
| (Glass Vial) | | | | | | | | | | | | |
| Sulfate | 1.6 | 98.1 | 0.3 | 1.2 | 98.5 | 0.3 | 1.2 | 98.3 | 0.5 | 1.4 | 97.9 | 0.6 |
| (Glass Vial) | | | | | | | | | | | | |
| Chloride | 1.2 | 98.5 | 0.3 | 2.2 | 97.5 | 0.3 | 4.0 | 95.5 | 0.4 | 4.9 | 94.6 | 0.5 |
| (Syringe) | | | | | | | | | | | | |
| Sulfate | 1.6 | 98.1 | 0.3 | 2.1 | 97.5 | 0.3 | 3.5 | 96.1 | 0.4 | 4.5 | 95.0 | 0.5 |
| (Syringe) | | | | | | | | | | | | |

### EXAMPLE 5

### Three Parameter, 2-Level Full Factorial (DOE #2)

A second DOE study can be performed to evaluate the effect of pH, arginine concentration, and protein concentration in citrate buffer. Table 14 shows the formulation conditions used in DOE #2.

TABLE 14
Formulation Conditions Used for the DOE #2 Study

| | Arginine | | Product |
|---|---|---|---|
| Sample | Concentration | | Concentration |
| No. | (mM) | pH | (mg/mL) |
| 1 | 200 | 5.0 | 2 |
| 2 | 200 | 6.0 | 8 |
| 3 | 200 | 6.0 | 2 |
| 4 | 200 | 6.0 | 8 |
| 5 | 250 | 5.5 | 6 |
| 6 | 250 | 5.6 | 5 |
| 7 | 260 | 6.6 | 5 |
| 8 | 300 | 6.0 | 6 |
| 9 | 300 | 6.0 | 2 |
| 10 | 300 | 6.0 | 8 |
| 11 | 300 | 6.0 | 2 |

bGCSF-T133-20K PEG can be dialyzed into 5 buffer conditions. Samples 1 and 2 can be dialyzed in a buffer containing 30 mM citrate and 200 mM arginine at pH 5.0. Samples 3 and 4 can be dialyzed in a buffer containing 30 mM citrate and 200 mM arginine at pH 5.0. Samples 5 and 6 can be dialyzed in a buffer containing 30 mM citrate and 250 mM arginine at pH 6.0. Samples 7 and 8 can be dialyzed in a buffer containing 30 mM citrate and 300 mM arginine at pH 6.0. Samples 9 and 10 can be dialyzed in a buffer containing 30 mM citrate and 300 mM arginine at pH 5.5. Each post-dialyzed sample can be adjusted to the final target product concentration and then divided into 2×1 mL aliquots in glass lyophilization vials to form two sets of samples: one set can be stored at 2-8° C. as controls and a second set can be stored at 40° C. for three days.

Table 15 shows SEC results. The change in aggregate is between −0.1% and 2.1%. Higher aggregate strongly correlates with higher product concentration. Delta depegylation is between 0.1% and 0.8%. Slightly higher depegylation correlates with low product concentration at low pH. As pH decreases, depegylation increases, and this trend is consistent with historical observations in the pre-formulation studies.

TABLE 15
Summary of SEC Results for DOE #2 Study

| | % Aggregate | | | % Monomer | | | % Depegylation | | |
|---|---|---|---|---|---|---|---|---|---|
| Run | | 40° C. | | | 40° C. | | | 40° C. | |
| # | Inital | for 3 Day | Difference | Inital | for 3 Day | Difference | Inital | for 3 Day | Difference |
| 1 | 1.0 | 1.2 | 0.2 | 98.7 | 97.8 | −0.8 | 0.3 | 1.0 | 0.7 |
| 2 | 0.8 | 2.8 | 2.0 | 98.9 | 96.6 | −2.3 | 0.3 | 0.7 | 0.4 |
| 3 | 1.4 | 1.3 | −0.1 | 98.4 | 98.3 | −0.1 | 0.2 | 0.4 | 0.2 |
| 4 | 0.9 | 2.6 | 1.7 | 98.8 | 97.1 | −1.8 | 0.2 | 0.3 | 0.1 |
| 5 | 0.9 | 2.0 | 1.1 | 98.8 | 97.4 | −1.4 | 0.3 | 0.6 | 0.3 |
| 6 | 1.0 | 2.0 | 1.0 | 98.7 | 97.4 | −1.3 | 0.3 | 0.6 | 0.3 |
| 7 | 1.0 | 1.8 | 0.8 | 98.8 | 97.7 | −1.1 | 0.3 | 0.6 | 0.3 |
| 8 | 1.1 | 1.2 | 0.1 | 98.6 | 97.7 | −0.9 | 0.3 | 1.1 | 0.8 |
| 9 | 0.7 | 2.8 | 2.1 | 99.0 | 96.4 | −2.6 | 0.3 | 0.8 | 0.5 |
| 10 | 1.2 | 1.2 | 0.0 | 98.6 | 98.4 | −0.2 | 0.2 | 0.5 | 0.2 |
| 11 | 0.9 | 2.2 | 1.3 | 98.8 | 97.3 | −1.5 | 0.3 | 0.4 | 0.1 |

### EXAMPLE 6

### Agitation Study

A forced agitation study can be performed to assess protein stability in the formulations. The samples can be prepared by dialyzing bGCSF-T133-20K PEG (16.6 mg/mL protein in 10 mM sodium acetate, 5% sorbitol pH 4.0) against 30 mM Citrate and 250 mM Arginine at pH 6.0. A portion of the dialyzed material can be diluted to a target concentration of 5 mg/mL using buffer 30 mM Citrate and 250 mM Arginine at pH 6.0. The pool cans then be filtered through a 0.22 micron filter and then be subjected to forced agitation in a glass beaker by mixing at 60 rpm using a magnetic stirrer for two hours at room temperature. Samples can be taken every 30 minutes.

All samples are clear, colorless, and free of visible particulates for all timepoints. The protein concentration, absorbance at 550 nm, and pH measurement for each timepoint are shown in Table 16.

TABLE 16
Summary of Protein Concentration, A₅₅₀, and pH Results from the
Agitation (Mixing) Study

| | A280 nm Assay | | | | |
|---|---|---|---|---|---|
| | Protein | Std. | | | |
| | Concentration | Dev. | RSD | A550 | |
| Sample | (mg/mL) | (%) | (%) | Absorbance | pH |
| T₀ | 4.9 | 0.03 | 0.60% | 0.00942 | 6.0 |
| T₃₀ₘᵢₙ Control | 5.0 | 0.01 | 0.30% | −0.00016 | 6.0 |
| T₃₀ₘᵢₙ Agitated | 4.9 | 0.04 | 0.70% | 0.00972 | 6.0 |
| T₆₀ₘᵢₙ Control | 4.9 | 0.03 | 0.60% | 0.00407 | 6.0 |
| T₆₀ₘᵢₙ Agitated | 4.9 | 0.02 | 0.40% | 0.03547 | 6.0 |
| T₉₀ₘᵢₙ Control | 5.0 | 0.05 | 1.00% | 0.09031 | 6.0 |
| T₉₀ₘᵢₙ Agitated | 4.9 | 0.03 | 0.60% | 0.08796 | 6.0 |
| T₁₂₀ₘᵢₙ Control | 5.0 | 0.03 | 0.60% | 0.08761 | 6.0 |
| T₁₂₀ₘᵢₙ Agitated | 4.9 | 0.03 | 0.60% | 0.08775 | 6.0 |

The protein concentration remains stable throughout the mixing duration. The pH remains consistent throughout the experiment. Product composition by SEC is also consistent throughout the study, as shown in Table 17. Overall, results from this study indicate that the protein is stable for the entire duration of forced agitation.

TABLE 17
Summary of SEC Results from the Agitation (Mixing) Study

| | % | % | % |
|---|---|---|---|
| Sample | Aggregate | PEG-bGCSF | bGCSF |
| T0 | 1.6 | 98.1 | 0.3 |
| T30min Control | 1.6 | 98.2 | 0.3 |
| T30min Agitated | 1.6 | 98.2 | 0.3 |
| T60min Control | 1.6 | 98.1 | 0.3 |
| T60min Agitated | 1.6 | 98.2 | 0.3 |
| T90min Control | 1.6 | 98.2 | 0.3 |
| T90min Agitated | 1.6 | 98.2 | 0.3 |
| T120min Control | 1.6 | 98.1 | 0.3 |
| T120min Agitated | 1.6 | 98.2 | 0.3 |

### EXAMPLE 7

### Freeze-Thaw Study

A freeze-thaw study can be performed to determine protein concentration and pH of various samples. Protein in the samples can be subjected up to five freeze and thaw cycles. Samples can be filtered through 0.22 micron filters and dispensed into 15 mL vials. One aliquot can be set aside as the control. For the remaining three aliquots, each freeze-thaw cycle could consist of freezing the protein solution for one hour at −75±5° C. and thawing at room temperature for approximately one hour until no ice is observed. The sample vial can be gently swirled three times to mix the sample. One aliquot can be set aside after the first, second, and fifth freeze and thaw cycles for testing.

All samples are clear, colorless, and free of visible particulates for all timepoints. The protein concentration, absorbance at 550 nm, and pH measurement for each timepoint are shown in Table 18.

TABLE 18
Summary of Protein Concentration, A₅₅₀, and pH Results from the
Freeze-Thaw Study

| | A280 nm Assay | | | | |
|---|---|---|---|---|---|
| | Protein | Std. | | | |
| | Concentration | Dev. | RSD | A550 | |
| Sample | (mg/mL) | (%) | (%) | Absorbance | pH |
| Cycle 0 | 20.6 | 0.5 | 2.20% | 0.11563 | 5.9 |
| Cycle 1 | 21.9 | 1.4 | 0.40% | 0.09471 | 5.9 |
| Cycle 2 | 22.6 | 0.3 | 1.40% | 0.12685 | 5.9 |
| Cycle 5 | 21.3 | 1.0 | 4.60% | 0.13357 | 5.9 |

The protein concentration remains stable after each freeze-thaw cycle. Furthermore, the pH remains consistent throughout the five freeze-thaw cycles. Product composition by SEC is similar across all timepoints and is shown in Table 19.

TABLE 19
Summary of SEC Results from the Freeze-Thaw Study

| | % | % | % |
|---|---|---|---|
| Sample | Aggregate | PEG-bGCSF | bGCSF |
| Cycle 0 | 1.6 | 98.1 | 0.3 |
| Cycle 1 | 1.5 | 96.1 | 0.4 |
| Cycle 2 | 1.6 | 98.0 | 0.4 |
| Cycle 5 | 1.5 | 96.1 | 0.4 |

### INCORPORATION OF SEQUENCE LISTING

Incorporated herein by reference in its entirety is the Sequence Listing for the above-identified Application. The Sequence Listing is disclosed on a computer-readable file and identified as follows: 20 kilobytes XML file named “70845-413493—Sequence Listing.xml”, created on Oct. 15, 2024.

1. A method of treating an animal having a disorder modulated by bG-CSF comprising a step of administering to the animal a therapeutically effective amount of the formulation comprising
(a) a bovine granulocyte colony stimulating factor (bG-CSF) polypeptide of SEQ ID NO: 1 or SEQ ID NO: 2 comprising a non-naturally encoded amino acid substituted at position 133 of SEQ ID NO: 1 or the corresponding amino acid in SEQ ID NO: 2; wherein the non-naturally encoded amino acid is para-acetylphenylalanine;
wherein the para-acetylphenylalanine is bonded to a water soluble polymer being a poly(ethylene) glycol moiety having a molecular weight of about 20 kDa;
(b) a citrate buffer;
(c) arginine, and optionally a counter ion for arginine;
(d) wherein the formulation contains less than 0.0033% of a surfactant; and
(e) wherein the pH of the formulation ranges from 5.7 to 6.6.

2. The method of claim 1, wherein the disorder is an infection.

3. The method of claim 1, wherein the infection is mastitis.

4. The method of claim 1, wherein the infection is shipping fever.

5. The method of claim 1, wherein the formulation is administered to the animal between two weeks and one day before giving birth.

6. The method of claim 1, wherein the formulation is administered to the animal on the day of giving birth or up to one week after giving birth.

7. The method of claim 1, wherein the animal is a periparturient cow.

8. The method of claim 1, wherein the formulation optionally includes one or more other therapeutic ingredients.

9. The method of claim 1, wherein the formulation is administered via oral, intravenous, intraperitoneal, intramuscular, transdermal, subcutaneous, topical, sublingual, intravascular, intramammary, or rectal means.

10. The method of claim 1, wherein the formulation is administered to the animal in an amount between about 0.5 and about 12 grams/liter.

11. A method of treating an animal having a disorder modulated by bG-CSF comprising a step of administering to the animal a therapeutically effective amount of a stable aqueous formulation comprising
(a) a bovine ganulocyte colony stimulating factor (bG-CSF) polypeptide of SEQ IDS NO: 1 or SEQ ID NO: 2 comprising a non-naturally encoded amino acid substituted at position 133 of SEQ ID NO: 1 or the corresponding amino acid in SEQ ID NO: 2, wherein the non-naturally encoded amino acid is para-acetylphenylalanine, wherein the para-acetylphenylalanine is bonded to a water soluble polymer being a poly(ethylene) glycol moiety;
(b) a citrate buffer;
(c) arginine, and optionally a counter ion for arginine;
(d) wherein pH of the formulation ranges from 5.7 to 6.6.

12. The method of claim 11, wherein the disorder is an infection.

13. The method of claim 11, wherein the infection is mastitis.

14. The method of claim 11, wherein the infection is shipping fever.

15. The method of claim 11, wherein the formulation is administered to the animal between two weeks and one day before giving birth.

16. The method of claim 11, wherein the formulation is administered to the animal on the day of giving birth or up to one week after giving birth.

17. The method of claim 11, wherein the animal is a periparturient cow.

18. The method of claim 11, wherein the formulation optionally includes one or more other therapeutic ingredients.

19. The method of claim 11, wherein the formulation is administered via oral, intravenous, intraperitoneal, intramuscular, transdermal, subcutaneous, topical, sublingual, intravascular, intramammary, or rectal means.

20. The method of claim 11, wherein the formulation is administered to the animal in an amount between about 0.5 and about 12 grams/liter.
